(19) United States
(12) Patent Application Publication
(10) Pub. No.: US 2015/0290362 A1
(43) Pub. Date: Oct. 15, 2015

(54) HYBRID FIBRIN-MICROGEL CONSTRUCTS FOR TISSUE REPAIR AND REGENERATION

(71) Applicant: Georgia Tech Research Corporation, Atlanta, GA (US)

(72) Inventors: Alison Douglas, Atlanta, GA (US); Thomas Barker, Decatur, GA (US); Louis Andrew Lyons, Marietta, GA (US); Alberto Fernandez-Nieves, Atlanta, GA (US)

(21) Appl. No.: 14/683,538

(22) Filed: Apr. 10, 2015

Related U.S. Application Data
(60) Provisional application No. 61/977,900, filed on Apr. 10, 2014.

(51) Int. Cl. A61L 27/54; A61L 27/44

(52) U.S. Cl. CPC A61L 27/54; A61L 27/44; A61L 2430/00

(57) ABSTRACT

Composite constructs of polymer matrices and polymeric microgels, and methods of making and using thereof, are described. The addition of the microgels to polymeric matrices retains the overall mechanical properties of the matrices and enables cell spreading, invasion and vascularization. Using fibrin as a model system, composite fibrin constructs with microgels are described, in which microgels form unique interconnected pockets within the fibrin matrix. The microgel assembly is driven by the polymerization dynamics of fibrin, and the architecture of these interconnected pockets can be tuned. The composite constructs support cell spreading, migration and infiltration more efficiently than do control matrices. The composite constructs can be used attract cells and/or to deliver therapeutic, diagnostic, nutraceutical, prophylactic and cosmetic agents to a desired site.

### CROSS REFERENCE TO RELATED APPLICATIONS

This application claims benefit of U.S. Provisional Application No. 61/977,900, filed Apr. 10, 2014, which is hereby incorporated herein by reference in its entirety.

### STATEMENT REGARDING FEDERALLY SPONSORED RESEARCH

This invention was made with government support under Grant Nos. EB011566 and EB013743 awarded by the National Institutes of Health. The government has certain rights in the invention.

### FIELD OF THE INVENTION

This invention is in the field of fibrin-microgel hybrid matrices for tissue remodeling and rapid wound healing that permit host cell infiltration.

### BACKGROUND OF THE INVENTION

Within the field of regenerative medicine, many tissue engineering approaches use natural protein-based polymer systems due to their enhanced endogenous bioactivity and potential for seamless integration with the host tissue. However, most natural biopolymers form weak hydrogels that do not fully match the mechanical properties of the target tissues making them difficult to apply in practice. High concentrations of protein are used to form more mechanically robust networks, but these networks have a smaller network mesh size due to the high concentrations of protein. Small network mesh size inhibits cellular processes such as spreading and migration—prerequisites for cell infiltration and biomaterial integration. Thus, there is a tension between selecting biomaterials with strong mechanical characteristics versus biomaterials with good cell invasion and tissue ingrowth.

There remains a need for improved biomaterials that can be formed both ex vivo and in vivo with mechanical properties that match those of tissues and permit cell invasion and cell ingrowth into the biomaterial.

Therefore, it is an object of the present invention to provide improved matrices for facilitating tissue remodeling while permitting host cell infiltration.

It is further object of the present invention to provide improved matrices wound healing while permitting host cell infiltration.

It is another object of the present invention to provide kits for assembling the improved matrices for tissue remodeling and rapid wound healing.

It is yet another object of the present invention to provide methods of making and using the improved matrices for tissue remodeling and rapid wound healing.

### SUMMARY OF THE INVENTION

Polymer matrices containing ultrasoft polymeric microgels, and methods of making and using them are described. The addition of ultrasoft polymeric microgels to dense polymeric matrices produces a composition that retains the overall mechanical properties of the polymer matrices and enables cell spreading, invasion and enhanced vascularization vis-a-vis the microgel. Using fibrin as a model system, fibrin matrices containing microgels are described in which microgels form unique interconnected structures within the fibrin matrix. The microgel assembly is driven by the polymerization dynamics of fibrin, and the architecture of these interconnected structures can be tuned. The composite constructs support cell spreading, migration and infiltration more efficiently than do control matrices. The composite constructs can be used attract cells and/or to deliver therapeutic, diagnostic, nutraceutical, prophylactic and cosmetic agents to a desired site.

One embodiment provides a composition having a polymeric matrix containing an ultralow crosslinked microgel. The composition preferably has a microgel volume fraction of 0.02 to 0.50. Still another embodiment has a microgel volume fraction of 0.02 to 0.168. The composition can be a composition in which a polymeric matrix is formed by polymerizing monomers in the presence of a microgel or microgels to produce a polymeric matrix that contains the microgel or microgels. In one embodiment, the microgel can be considered to be suspended in the polymeric matrix.

The microgel can heterogeneously or homogeneously dispersed within the polymer matrix. In certain embodiments, the microgels segregate into pockets within the polymer matrix. Said pockets may or may not be interconnected.

A preferred polymeric matrix contains fibrin. A preferred microgel contains poly(N-isopropylacrylamide) (pNIPAm), polyketal polymers, poly(ethylene glycol) or poly[(oligoethylene glycol)methacrylate].

The composition can be loaded with therapeutic agents and substances including, but not limited to cells, growth factors, cytokines, chemokines, anti-inflammatory agents, angiogenesis factors, proteins, or drugs.

Still another embodiment provides a method of repairing or regenerating a tissue in a subject by administering to the subject an effective amount of the polymeric matrix containing the microgel, wherein the microgel-polymer composite contains a therapeutic agent or is seeded with human cells.

### BRIEF DESCRIPTION OF THE DRAWINGS

FIG. 1 is a diagram of formation of microgels via precipitation polymerization from a growing polymer chain

FIG. 2A is a graph showing change in microgel radius (R [nm]) as a function of temperature (° C.) as measured by static (R_{g}) or dynamic (Rₕ) light scattering. The microgels were formed via precipitation polymerization using either BIS as a crosslinker, or no crosslinker to form ultra-low crosslinked microgels (ULC). FIG. 2B is a graph showing change in the ratio of Rg/Rh as a function of temperature (° C.) in a pH 7.4 buffer containing 150 mM NaCl.

FIG. 3 is a graph showing the viscometry curves for determination of the constant, k, for calculating microgel volume fraction. Dynamic viscosities of the microgel suspensions at various concentrations and temperatures were calculated from time measurements and fit to the Einstein-Batchelor equation.

FIGS. 4A, 4B and 4C are graphs showing the storage or loss moduli of fibrin-microgel composites when microgels are incorporated into fibrin gels. FIG. 4A is a graph of the average storage modulus (G′, Pa) for fibrin matrix and a fibrin matrix incorporating microgels when 8 mg/mL fibrin matrices were polymerized±ULC Φ=0.112 (n=6), with standard deviation (p-value 0.37). FIG. 4B is a graph of the average storage modulus (G′, kPa) of fibrin gel or fibrin and microgel composites of different fibrin concentrations. FIG. 4C is a graph of a representative G′G″ curve of the control (fibrin) and experimental (fibrin and microgel) groups.

FIGS. 5A, 5B and 5C are graphs showing the effect of microgel volume fraction on NIH3T3 fibroblast cell area (μm², FIG. 5A), on circularity (FIG. 5B), and elliptical factor (FIG. 5C) during spreading of the cells in fibrin-microgel composites containing different concentrations of microgel (Φ). The cells, stained for actin, were analyzed for cell area, circularity (=4*π*Area/Perimeter²), and elliptical factor (major/minor axis), which were measured from at least 50 different cells in 3 different gels. (Box and whiskers plots show 95-5% confidence intervals, ***p<0.001, ANOVA).

FIGS. 6A and 6B are graphs showing cell migration velocity (μm/hour) of NIH3T3 fibroblast cells in fibrin-microgel composites containing different concentrations of microgel (Φ). Velocity was calculated from individual cells encapsulated within gel constructs over the course of 12 hours. Box and whiskers plots show 95-5% confidence intervals, ***p<0.05, ANOVA.

FIG. 7 is a graph showing migration distance (μm) at 1, 2, and 3 days in culture of NIH3T3 cells as the cells invade surrounding gel without (black) and with Φ=0.112 (gray) microgels. Migration distance was measured from 10× phase contrast images taken each day in 12 gels over two separate experiments. Box and whiskers plot p<0.001 Two-way ANOVA.

FIGS. 8A, 8B and 8C are graphs showing the vascular volume fraction (vascular volume (VV) over total volume (TV), VV/TV, FIG. 8A), vascular volume (mm³, FIG. 8B), and total volume (mm³, FIG. 8C) calculated for the subcutaneously implanted constructs containing ULC microgels (φ=0.112) in fibrin at 8 mg/mL or 25 mg/mL or only fibrin at 8 mg/ml, 25 mg/ml or 2.5 mg/ml, and polymerized in polycaprolactone (PCL) nanofiber meshes. Going from left to right for each graph is fibrin at 8 mg/mL (red) or 25 mg/mL (purple) or only fibrin at 8 mg/ml (blue), 25 mg/ml (green) or 2.5 mg/ml (orange), and polymerized in polycaprolactone (PCL) nanofiber meshes.

FIGS. 9A, 9B, and 9C are graphs showing the vessel arc length (mm, FIG. 9A), total vessel volume (mm³, FIG. 9B), and total number of branches (#, FIG. 9C) of microvessels isolated from rat adipose tissue embedded in constructs containing 2.5 mg/ml, 8 mg/ml, or 25 mg/ml fibrin with or without 4 mg/ml (φ=0.112) ULC microgels.

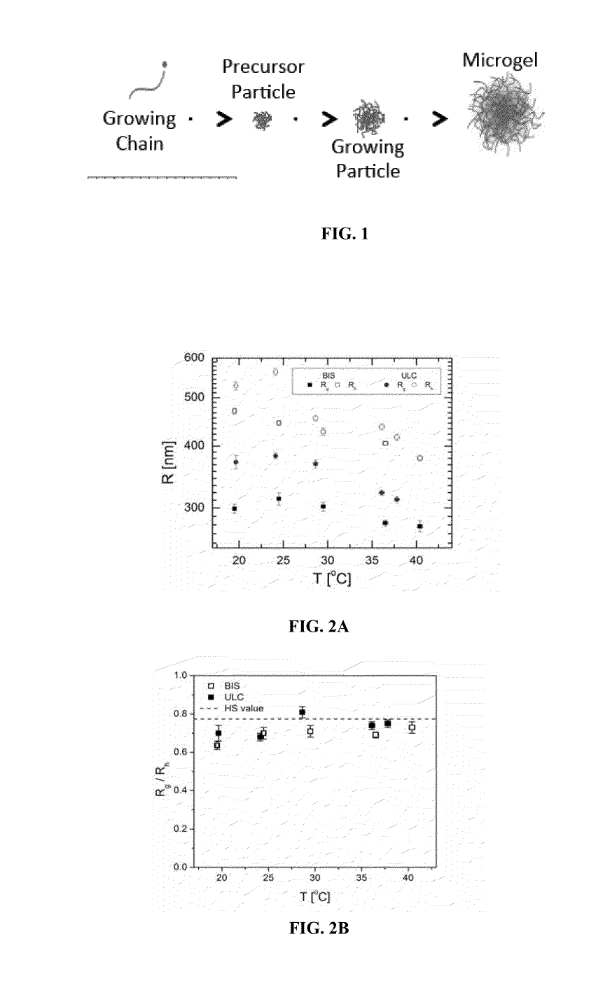

### DETAILED DESCRIPTION OF THE INVENTION

### I. Definitions

As used herein, “polymer” refers to a molecule consisting of a number of repeating units.

As used herein, “repeat unit” refers to the fundamental recurring unit of a polymer.

As used herein, “monomer” refers to the smaller molecule(s) that are used to prepare a polymer. It may or may not be equivalent to the repeat unit.

As used herein, “macromer” and “macromonomer” refers to any polymer or oligomer that has a functional group that can take part in further polymerization.

As used herein, “microgel” refers to a solvent swollen colloidal particle fonned from a crosslinked network of polymer or macromer chains.

As used herein, “subject or patient” refers to a mammal, primate and preferably a human.

As used herein, the term “matrix” refers to polymerized monomers of a polymer that is chemically or physically crosslinked to form a 3-dimensional network.

As used herein, the term “matrix-microgel composite” refers to a matrix wherein microgels are suspended within that matrix.

As used herein, the term “pocket” refers to the microgel-rich regions and matrix-poor regions of the matrix-microgel composite. The matrix and the microgel are not covalently bonded to each other.

### II. Compositions

One embodiment provides a polymeric matrix composition containing one or more ultralow crosslinked microgels. The compositions can be used in tissue regeneration, tissue engineering, wound care, and as delivery vehicles to deliver agents to the sites of administration. Also the compositions may incorporate progenitor cells, and/or biological molecules that promote infiltration and migration of cells into the assembly or secrete or produce factors to alter the microenvironment at the site of administration to promote or enhance tissue regeneration or wound repair.

A. Polymeric Matrix.

Typically, the polymeric matrix is formed of biocompatible polymer, protein, polysaccharide, or another polymer that is able to replicate the mechanical and structural properties of biological tissues. In preferred embodiments, the polymer matrix is formed of extracellular matrix proteins, such as fibrin, or collagen. In other embodiments, the polymer matrix is formed of polysaccharides, or biocompatible polymers.

### 1. Extracellular Matrix Components

Typically, the proteins used to form the polymeric matrix are extracellular matrix (ECM) components. These include, but are not limited to, collagen, elastin, fibronectin, fibrin, and laminin

The essential non-cellular component of all tissues is the ECM, the protein scaffold and microenvironment in which cells reside. The protein composition and structure of the ECM can influence cell adhesion and signaling, as cells bind to specific ligand sites on ECM proteins via cell surface receptors, including integrins and proteoglycans. Cell-ECM interactions are complex and dynamic in nature and can dictate cellular phenotype, influencing cell survival, proliferation, migration, and differentiation. Various ECM proteins also bind and sequester growth factors, which can lead to spatial and temporal control of the dynamics of cellular processes. Additionally, the ECM can determine the mechanical properties of each organ through its compressive modulus, elasticity, and water retention, which can play a protective role in tissues to maintain homeostasis.

There are several canonical ECM proteins that are prevalent throughout many tissues in the body, each with their own complexities. Most tissues are heterogeneous mixtures of various proteins including collagens, laminins, and proteoglycans among others. A primary function of ECM proteins is to facilitate cell adhesion, in which they serve as ligands for one or many of the heterodimeric transmembrane cell surface receptors, termed integrins.

While most ECM proteins are slowly assembled and synthesized by cells within the surrounding tissue, an exception is fibrin, which is formed quickly through the initiation of the coagulation cascade. During this process, polymerization of soluble fibrinogen, initiated by thrombin cleavage and activation, forms an insoluble fibrin clot or matrix. It is also important to note that the ECM is naturally in a non-equilibrium state as cells are constantly remodeling the network through deformations induced by cell contractile forces and degradation induced by numerous proteases, including the matrix metalloproteinases (MMPs), in addition to synthesizing new protein components within the tissue structure.

Therefore, the native ECM is an extremely complex, dynamic environment through which many cell fate processes are controlled. The fields of tissue engineering and regenerative medicine have sought to develop biomaterials capable of recapitulating the behavior of the ECM in the context of synthetic or biosynthetic hybrids of reduced complexity, with the ability to regenerate severely damaged tissues either in vitro or in vivo remaining the primary goal. In addition, such materials can lead to the creation of accurate models to study cell behavior and fate under a multitude of environmental conditions. Hydrogels, both synthetic and natural, have been a main choice for ECM mimics because their mechanical properties and high water content resemble those of many tissues.

Traditional synthetic hydrogels exhibit tunable mechanical properties that are similar to that of many tissues, making them widely studied for tissue engineering and regenerative medicine applications. Reductionist ECMs, like collagen or fibrin gels, are useful for soft tissue applications where the elastic modulus needs to be on the order of Pascals to several kiloPascals. However, for tissues like bone, cartilage, and muscle, hundreds of kPa are required for proper cell growth, phenotype maintenance, and tissue development. A common issue in reductionist and synthetic ECM approaches is that the gels are often not mechanically robust enough for load-bearing tissue applications. One of the primary challenges remaining in tissue engineering and regenerative medicine is the development of biomaterials that display robust mechanical properties while simultaneously being permissive to cellular invasion. It is currently typical that one can optimize either feature independently, but optimization of both simultaneously represents a significant challenge. A possible solution to increase the elastic modulus is to increase the polymer concentration or the number of crosslinks in the gel. However, this approach can be limiting, as simultaneous changes in gel porosity and, in some cases, ligand density influence material mechanical properties, cell migration and diffusion of molecules within the gel. For example, the ability to increase in the modulus of poly(ethylene glycol) (PEG) hydrogels without affecting mesh size has been demonstrated; however, the mesh size is still on the order of tens of nanometers, which is not cell permissive.

An alternative approach has been to develop synthetic additives that can increase the elastic modulus of hydrogels. This has been accomplished through the development of double network hydrogels and colloid reinforced hydrogels. Haraguchi et al. demonstrated the reinforcement of a poly(N-isopropylacrylamide) hydrogel by using inorganic clay disks as the crosslinking points within the gel. A similar effect has been observed in poly(ethylene oxide) hydrogels. Additional approaches regarding nanoparticle-based reinforcement in composite materials have also been reported. In these nanocomposite gels, particles act as cross-linkers within the bulk gel, enhancing the mechanical toughness beyond that expected for traditional organic cross-linkers. Importantly, the presence of particulate cross-linkers in these cases creates more porous networks unlike their small molecule counterparts, which decreases the pore size of the gel as the cross-linker concentration increases.

While hard, inorganic particles have been successful in enhancing the elastic modulus of hydrogels, soft hydrogel colloids have also become a promising route. A study reported by Richtering and co-workers demonstrated that temperature-responsive hydrogel microparticles (microgels) simply embedded in a hydrogel matrix resulted in a modest increase in the elastic and storage moduli and the strain and stress at break. Temperature induced de-swelling of the particles resulted in a transformation from soft to hard particles, further enhancing the mechanical properties of the composite gel. An additional consequence of the induced volume change is the generation of a matrix with switchable porosities. Saunders and co-workers have reported enhanced mechanical properties by chemically crosslinking stimuli-responsive microgels into a hydrogel network Importantly, these doubly cross-linked microgel networks were shown to function in a potentially load-bearing application: the reconstruction of deteriorated intervertebral discs. Degenerated discs injected with doubly cross-linked microgels had mechanical properties similar to those of normal intervertebral discs. The authors proposed that the doubly cross-linked microgel network increased the swelling pressure within the discs because of the high water content, permitting the discs to behave in a manner similar to the natural system of collagen and proteoglycans: collagen is the structural unit and the highly charged proteoglycans provide internal swelling osmotic pressure.

Proteoglycans are major components of many ECMs, where they act as fillers, contribute elasticity within connective tissues, and are important in molecular transport through the ECM. Proteoglycans have a protein backbone with one or more glycosaminoglycans or linear carbohydrate polymers covalently attached to the core protein. They are often sulfated, rendering them negatively charged under physiological conditions. This charge allows them to mediate signaling and regulate the movement of molecules through the matrix by binding cations and water molecules. Similarly, microgels are highly swollen with water and can be synthesized to contain ionizable groups within the network. Microgels, therefore, may be able to recapitulate the characteristics of proteoglycans in artificial ECMs.

Synthetic systems lack essential ECM components, like integrin ligands, to foster cell binding and proliferation, and, therefore, require modification to include these molecules. Additionally, they must be rendered degradable to be fully integrated into a biological system. Natural ECM polymers (e.g. collagen, fibrin, hyaluronic acid, fibronectin, laminin) have cell adhesive domains present for binding, and can be degraded and remodeled using enzymes secreted by resident cells. Colloids have the potential to provide a means of tailoring the mechanical properties of biologically derived hydrogels, which are easier to integrate into tissues than purely synthetic architectures.

Fibrin is an attractive base polymer for use in engineering wound repair, as it forms the provisional scaffold during initiation of endogenous tissue repair processes. Not only is it fundamental in hemostasis, but it also serves as a biologic scaffold that binds a multitude of factors, concentrating them to the wound microenvironment to enhance subsequent repair of the damaged tissue. Fibrin and its precursor, fibrinogen, present binding sites for other pro-coagulant proteins (thrombin and Factor XIII), pro-fibrinolytic proteins (plasminogen, tissue plasminogen activator), anti-fibrinolytic proteins (plasminogen activator inhibitor), growth factors (VEGF, PDGF, bFGF), ECM glycoproteins (fibronectin, heparin), and cell surface receptors (integrins, cadherins). All of these components make up a network that is capable of transitioning the provisional wound matrix into a healed tissue structure. During tissue repair, invading cells are able to migrate into the fibrin matrix and degrade/remodel the polymer over time. However, if fibrin deposition and degradation processes are imbalanced (e.g. due to supra-physiological concentrations of fibrinogen), then cellular infiltration, ECM remodeling, and regenerative processes are hindered. Fibrinogen can be conveniently isolated from serum and is used clinically as a tissue sealant in combination with its activator, thrombin. It has also been extensively studied as a tissue construct for regeneration of many tissues including cardiac, muscle, skin, vascular and bone. Current FDA-approved fibrin sealants on the market such as Tisseel® (Baxter Healthcare) and Evicel (Johnson & Johnson) typically contain supra-physiological fibrinogen concentrations ranging from 20-100 mg/mL, while the native human clot is approximately 2-4 mg/mL. Mechanical and diffusional limitations of these supra-physiological clots impede cellular infiltration and remodeling. However, in order to generate fibrin gels with mechanical properties similar to osteoid for bone repair applications, for example, high concentrations of protein are necessary, resulting in networks that are extremely dense and exhibit nanoscale pores. This can lead to poor regeneration of bone tissue in vivo. The same issues hold for fibrinogen based tissue sealants that are used at high concentrations to facilitate rapid wound closure, but then subsequently prevent host cell infiltration and remodeling, which in turn prevents complex morphogenic processes, such as angiogenesis, during tissue remodeling or would healing.

Collagens are the most abundant protein in the ECM. Collagens are present in the ECM as fibrillar proteins and give structural support to resident cells. Collagen is exocytosed in precursor form (procollagen), which is then cleaved by procollagen proteases to allow extracellular assembly.

Elastins, in contrast to collagens, give elasticity to tissues, allowing them to stretch when needed and then return to their original state. This is useful in blood vessels, the lungs, in skin, and the ligamentum nuchae, and these tissues contain high amounts of elastins. Elastins are synthesized by fibroblasts and smooth muscle cells. Elastins are highly insoluble, and tropoelastins are secreted inside a chaperone molecule, which releases the precursor molecule upon contact with a fiber of mature elastin. Tropoelastins are then deaminated to become incorporated into the elastin strand.

Fibronectins are glycoproteins that connect cells with collagen fibers in the ECM, allowing cells to move through the ECM. Fibronectins bind collagen and cell-surface integrins, causing a reorganization of the cell's cytoskeleton and facilitating cell movement. Fibronectins are secreted by cells in an unfolded, inactive form. Binding to integrins unfolds fibronectin molecules, allowing them to form dimers so that they can function properly. Fibronectins also help at the site of tissue injury by binding to platelets during blood clotting and facilitating cell movement to the affected area during wound healing.

Laminins are proteins found in the basal laminae of virtually all animals Rather than forming collagen-like fibers, laminins form networks of web-like structures that resist tensile forces in the basal lamina. They also assist in cell adhesion Laminins bind other ECM components such as collagens, nidogens, and entactins.

### 2. Polysaccharides

Optionally, the polymeric matrix is formed of cross-linkable polysaccharides. Suitable polysaccharides include alginate, chitosan, glucosaminoglycans, and plant- or fungal-derived compounds, including pectins, galactomannans/mannoglycans, xyloglucans, and beta-glucans/lentinans. Other polysaccharides include chitosan, fucoidan, galactan, carrageenan, k-carrageenan, galactofucan, mannoglucoronofucan, arabinogalactans, xylomannan sulfate, xylogalactofucan, ulvan, dextrans and derivatives thereof, and other compounds such as described by Chattopadhyay, International Journal of Polymer Science, 2010, 2010:1-7; or Patel, 3 Biotech, 2012, 2:171-185).

Chondroitin sulfate (formerly called a mucopolysaccharide) is found in cartilage, bone, blood vessels and connective tissues. There are two forms: chondroitin sulfate A and chondroitin sulfate C. One or both types accumulate abnormally in several of the mucopolysaccharidosis disorders. Chondroitin sulfate contains N-acetyl galactosamine alternating with glucuronic acid to form the disaccharide repeating unit of polymer. Chondroitin sulfate consists of a chain of about 40 repeating units of N-acetyl chondrosine sulfate with about 80 anionic charges. Chondroitin sulfate is the most prevalent of the glycoaminoglycan in cartilage. Chondroitin sulfate B is called dermatan sulfate.

Dermatan sulfate (formerly called a mucopolysaccharide) is found mostly in skin but also in blood vessels, the heart valves, tendons, and the lungs. Dermatan sulfate accumulates abnormally in several of the mucopolysaccharidosis disorders.

Keratin sulfate is a disaccharide repeating unit consisting of N-acetyl glucosamine alternating with galactose. It has variable chain length and variable degree of sulfonation. Keratin sulfate is present in low levels in fetal & newborn cartilage, but the concentration rises with maturation up to 55% of total glycosaminoglycan content of the tissue. Morquio's Syndrome is a disorder characterized by excessive accumulation of keratin sulfate in the tissues.

Hyaluronic acid (hyaluronan or hyaluronate) is an anionic non-sulfated GAG. The term hyaluronate also refers to the conjugate base of hyaluronic acid. Because the molecule typically exists in vivo in its polyanionic form, it is most commonly referred to as hyaluronan It is a visco-elastic polymer normally found in the aqueous and vitreous humour. Sodium hyaluronate (ORTHOVISC®) is a viscous solution consisting of a high molecular weight (500-700 kDa) fraction of purified natural sodium hyaluronate in buffered physiological sodium chloride. Hyaluronic acid is a natural complex sugar of the glycosaminoglycan family and is a long-chain polymer containing repeating disaccharide units of Na-glucuronate-N-acetylglucosamine. Sodium hyaluronate occurs naturally on the corneal endothelium, bound to specific receptors for which it has a high affinity. It is also used to treat knee pain in patients with joint inflammation (osteoarthritis). It is usually used in patients who have not responded to other treatments such as acetaminophen, exercise, or physical therapy. Sodium hyaluronate may also be used in plastic surgery to reduce wrinkles on the face or as a filler in other parts of the body. It may be used in ophthalmology to assist in the extraction of cataracts, the implantation of intraocular lenses, corneal transplants, glaucoma filtration, retinal attachment and in the treatment of dry eyes. Finally, sodium hyaluronate is also used to coat the bladder lining in treating interstitial cystitis. Hyaluronan is similar to a substance that occurs naturally in the joints. It may work by acting as a lubricant and shock absorber in the joint, helping the knee to move smoothly, thereby lessening pain.

Rhamnan sulfate is a naturally occurring rhamnose-containing sulfated polysaccharide with antioxidant, antitcoagulant and antiviral biological activities. It is extracted from marine red, green and brown seaweeds (Patel, Therapeutic importance of sulfated polysaccharides from seaweeds: updating the recent findings. 3 Biotech, 2012, 2:171-185; Harada and Maeda, Chemical structure of antithrombin-active rhamnan sulfate from Monostrom nitidum. Bioscience, Biotechnology, and Biochemistry, 1998, 62:1647-1652). In certain embodiments, active agents for use with organophosphates include polysaccharides of plant, fungal or animal origin, such as plant- or fungal-derived pectins, galactomannans/mannoglycans, xyloglucans, and beta-glucans/lentinans. In certain embodiments, polysaccharides also include chitosan, alginate, fucoidan, galactan, carrageenan, k-carrageenan, galactofucan, mannoglucoronofucan, arabinogalactans, xylomannan sulfate, xylogalactofucan, dextran and derivatives thereof and ulvan.

Mannan from Candida albicans exhibits certain immunomodulatory properties. In general, these compounds consist mostly of a polysaccharide component but also include proteins (5% by weight). Mannose-binding lectins present on macrophages can bind mannan and activate the host immune system via a nonself-recognition mechanism (Tzianabos, Clinical Microbiology Reviews, 2000, 523-533).

Xyloglucans are polysaccharides that occur in the primary cell walls of all angiosperms (flowering plants) and medicinal mushrooms. Xyloglycans isolated from medicinal mushrooms exhibit antitumor effects (Walser et al., Critical Reviews in Immunology, 1999, 19:65-96).

Alginate exhibits high capacity for water absorption and is capable of absorbing 200-300 times its own weight in water. Alginate is a linear copolymer with homopolymeric blocks of mannuronate and guluronate residues. Alginate is used in various pharmaceutical preparations as an inactive ingredient, such as in Gaviscon, Bisodol, and Asilone. Alginate is used extensively as an impression-making material in dentistry, prosthetics, lifecasting and occasionally for creating positives for small-scale casting. Alginate is also used as an additive in dehydrated products for slimming aids, and is used by the weight loss industry as an appetite suppressant. Chitosan is a nontoxic and biodegradable polymer that has antibacterial, antiviral, antacid properties. Chitosan can also be used for film or fiber formation, or for forming hydrogels. (Chattopadhyay et al., International Journal of Polymer Science, 2010, 2010:1-7).

Fucoidan has been noted for antioxidant, immunostimulatory, lipid lowering, antibacterial and antihyperpeisic effects. Fucoidan and ulvan are also used in nanomedicine for wound healing, and for in vitro and in vivo controlled drug release (Patel, 3 Biotech, 2012, 2:171-185).

Galactan, carrageenan and k-carrageenan exhibit antioxidant, immunostimulatory, anti-inflammatory and antinociceptive, anticoagulant and antiviral effects. Galactofucan and mannoglucoronofucan may have antitumor effects. Arabinogalactants may have anticoagulant and antithrombotic effects. Xylomannan sulfate and xylogalactofucan exhibit antiviral effects, particularly against such viruses as influenza, herpes and human immunodeficiency virus (Patel, 3 Biotech, 2012, 2:171-185).

Dextran is a branched polysaccharide. Both dextran and many of its naturally-occurring and synthetic derivatives exhibit antithrombic activity.

### 3. Biocompatible Polymers

Representative biocompatible polymers include, but are not limited to polyethylene glycol) based polymers; poly[(oligo ethyleneglycol) methacrylate] based polymers, and combinations thereof.

### 4. Crosslinkers

The biological or biocompatible polymers form the polymer matrix through crosslinking of individual polymer strands together. The crosslinking can be achieved with biological crosslinkers and/or clotting factors that activate polymerization or chemical crosslinking.

### a. Biological Crosslinkers, Activators of Polymerization, Clotting Factors

The crosslinking of biological or biocompatible polymers form the polymeric network can be achieved through biological crosslinking and/or clotting. Biological crosslinkers include structural or enzymatic molecules with high (<10⁻⁹ M) affinities for their substrates. Examples include crosslinking of the monomers and polymers through the biotin-avidin molecular interactions, or through proteins A, L, G interactions with immunoglobulin heavy and light chains. Other examples include enzymes that catalyze crosslinking, such as Factor XIIIa, or proteolytic activations, such as thrombin.

In preferred embodiments, the proteolytica activator thrombin is used.

### i. Thrombin

Thrombin is formed from the proteolytic cleavage of prothrombin (coagulation factor II) of the coagulation cascade. Formation of thrombin ultimately results in the reduction of blood loss. Thrombin acts as a serine protease that converts inactive soluble fibrinogen into active fibrin monomer that self-assembles into insoluble strands of fibrin, as well as catalyzing many other coagulation-related reactions.

In the blood coagulation pathway, thrombin acts to convert factor XI to XIa, VIII to VIIIa, V to Va, fibrinogen to fibrin, and XIII to XIIIa. Factor XIIIa is a transglutaminase that catalyzes the formation of covalent bonds between lysine and glutamine residues in fibrin or fibrinogen. The covalent bonds increase the stability of the fibrin clot.

### b. Chemical Crosslinkers

Chemical crosslinkers that can be used to crosslink polymers, proteins, peptides, polysaccharides, etc. are known in the art and are defined based on utility and structure and include DSS (Disuccinimidylsuberate), DSP (Dithiobis(succinimidylpropionate)), DTSSP (3,3′-Dithiobis (sulfosuccinimidylpropionate)), SULFO BSOCOES (Bis[2-(sulfosuccinimdooxycarbonyloxy) ethyl]sulfone), BSOCOES (Bis[2-(succinimdooxycarbonyloxy)ethyl]sulfone), SULFO DST (Disulfosuccinimdyltartrate), DST (Disuccinimdyltartrate), SULFO EGS (Ethylene glycolbis(succinimidylsuccinate)), EGS (Ethylene glycolbis(sulfosuccinimidylsuccinate)), DPDPB (1,2-Di[3′-(2′-pyridyldithio) propionamido]butane), BSSS (Bis(sulfosuccinimdyl) suberate), SMPB (Succinimdyl-4-(p-maleimidophenyl) butyrate), SULFO SMPB (Sulfosuccinimdyl-4-(p-maleimidophenyl) butyrate), MBS (3-Maleimidobenzoyl-N-hydroxysuccinimide ester), SULFO MBS (3-Maleimidobenzoyl-N-hydroxysulfosuccinimide ester), SIAB (N-Succinimidyl(4-iodoacetyl) aminobenzoate), SULFO SIAB (N-Sulfosuccinimidyl(4-iodoacetyl)aminobenzoate), SMCC (Succinimidyl-4-(N-maleimidomethyl) cyclohexane-1-carboxylate), SULFO SMCC (Sulfosuccinimidyl-4-(N-maleimidomethyl) cyclohexane-1-carboxylate), NHS LC SPDP (Succinimidyl-6-[3-(2-pyridyldithio) propionamido) hexanoate), SULFO NHS LC SPDP (Sulfosuccinimidyl-6-[3-(2-pyridyldithio) propionamido) hexanoate), SPDP (N-Succinimdyl-3-(2-pyridyldithio) propionate), NHS BROMOACETATE (N-Hydroxysuccinimidylbromoacetate), NHS IODOACETATE (N-Hydroxysuccinimidyliodoacetate), MPBH (4-(N-Maleimidophenyl) butyric acid hydrazide hydrochloride), MCCH (4-(N-Maleimidomethyl) cyclohexane-1-carboxylic acid hydrazide hydrochloride), MBH (m-Maleimidobenzoic acid hydrazidehydrochloride), SULFO EMCS (N-(epsilon-Maleimidocaproyloxy) sulfosuccinimide), EMCS (N-(epsilon-Maleimidocaproyloxy) succinimide), PMPI (N-(p-Maleimidophenyl) isocyanate), KMUH (N-(kappa-Maleimidoundecanoic acid) hydrazide), LC SMCC (Succinimidyl-4-(N-maleimidomethyl)-cyclohexane-1-carboxy(6-amidocaproate)), SULFO GMBS (N-(gamma-Maleimidobutryloxy) sulfosuccinimide ester), SMPH (Succinimidyl-6-(beta-maleimidopropionamidohexanoate)), SULFO KMUS (N-(kappa-Maleimidoundecanoyloxy)sulfosuccinimide ester), GMBS (N-(gamma-Maleimidobutyrloxy) succinimide), DMP (Dimethylpimelimidate hydrochloride), DMS (Dimethylsuberimidate hydrochloride), MHBH (Wood's Reagent; Methyl-p-hydroxybenzimidate hydrochloride, 98%), DMA (Dimethyladipimidate hydrochloride).

B. Microgels

### 1. Polymers for Microgels

Microgels may be formed from any polymer capable of being formed into a cross-linked network. Since microgels are colloidal particles, it is the synthesis method that distinguishes microgels from macroscopic bulk polymer networks. Polymer networks can be synthesized into the form of microgels via a number of synthetic approaches, including emulsion, microemulsion, miniemulsion, precipitation, and template driven polymerization. Microgels may also be formed via the assembly and crosslinking of pre-formed polymers such as polysaccharides.

### a. p(N-isopropylacrylamide-co-acrylic-acid)

Microgels formed of pNIPAm are desirable for their thermosensitive properties (Lyon and Fernandez-Nieves, Annu. Rev. Phys. Chem. 63:25-43 (2012), which is incorporated herein by reference in its entirety).

Lyon and Fernandez-Nieves (2012) report that pNIPAm microgels are thermosensitive particles displaying a volume phase transition temperature (VPTT) of ˜32° C.; and deswell with increasing temperature. The VPTT of the microgels is directly associated with the lower critical solution temperature (LCST) of the polymer comprising the microgel. The two terms are often used interchangeably, given that they coincide for pure pNIPAm microgels. However, the VPTT is the temperature associated with the thermally induced collapse of the microgel, whereas the LCST is related to the thermally induced desolvation of the polymer chains; the two values are related but not necessarily identical. Nonetheless, the thermally induced desolvation of microgels provides a route through which Φ can be changed by controlling the ambient temperature. Below the VPTT, for a certain temperature range, the interaction between particles can be approximated by a hard-sphere interaction. Even if there is charge on the periphery of the particles, when the particles are very swollen, the charge density in this region is very small; electrostatic interactions are thus small when compared to the impact of physical volume exclusion between microgels. In addition, because the particles contain a large amount of solvent in this temperature range, van der Waals interactions between them are also negligible. The interaction potential and the phase behavior are thus reminiscent of the phase behavior of hard spheres.

Optionally, the microgels are formed of cross-linkable polysaccharides. Suitable polysaccharides include alginate, chitosan, glucosaminoglycans, and plant- or fungal-derived compounds, including pectins, galactomannans/mannoglycans, xyloglucans, and beta-glucans/lentinans. Other polysaccharides include chitosan, fucoidan, galactan, carrageenan, k-carrageenan, galactofucan, mannoglucoronofucan, arabinogalactans, xylomannan sulfate, xylogalactofucan, ulvan, dextrans and derivatives thereof, and other compounds such as described by Chattopadhyay, International Journal of Polymer Science, 2010, 2010:1-7; or Patel, 3 Biotech, 2012, 2:171-185).

Chondroitin sulfate (formerly called a mucopolysaccharide) is found in cartilage, bone, blood vessels and connective tissues. There are two forms: chondroitin sulfate A and chondroitin sulfate C. One or both types accumulate abnormally in several of the mucopolysaccharidosis disorders. Chondroitin sulfate contains N-acetyl galactosamine alternating with glucuronic acid to form the disaccharide repeating unit of polymer. Chondroitin sulfate consists of a chain of about 40 repeating units of N-acetyl chondrosine sulfate with about 80 anionic charges. Chondroitin sulfate is the most prevalent of the glycoaminoglycan in cartilage. Chondroitin sulfate B is called dermatan sulfate.

Dermatan sulfate (formerly called a mucopolysaccharide) is found mostly in skin but also in blood vessels, the heart valves, tendons, and the lungs. Dermatan sulfate accumulates abnormally in several of the mucopolysaccharidosis disorders.

Keratin sulfate is a disaccharide repeating unit consisting of N-acetyl glucosamine alternating with galactose. It has variable chain length and variable degree of sulfonation. Keratin sulfate is present in low levels in fetal & newborn cartilage, but the concentration rises with maturation up to 55% of total glycosaminoglycan content of the tissue. Morquio's Syndrome is a disorder characterized by excessive accumulation of keratin sulfate in the tissues.

Hyaluronic acid (hyaluronan or hyaluronate) is an anionic non-sulfated GAG. The term hyaluronate also refers to the conjugate base of hyaluronic acid. Because the molecule typically exists in vivo in its polyanionic form, it is most commonly referred to as hyaluronan. It is a visco-elastic polymer normally found in the aqueous and vitreous humour. Sodium hyaluronate (ORTHOVISC®) is a viscous solution consisting of a high molecular weight (500-700 kDa) fraction of purified natural sodium hyaluronate in buffered physiological sodium chloride. Hyaluronic acid is a natural complex sugar of the glycosaminoglycan family and is a long-chain polymer containing repeating disaccharide units of Na-glucuronate-N-acetylglucosamine. Sodium hyaluronate occurs naturally on the corneal endothelium, bound to specific receptors for which it has a high affinity. It is also used to treat knee pain in patients with joint inflammation (osteoarthritis). It is usually used in patients who have not responded to other treatments such as acetaminophen, exercise, or physical therapy. Sodium hyaluronate may also be used in plastic surgery to reduce wrinkles on the face or as a filler in other parts of the body. It may be used in ophthalmology to assist in the extraction of cataracts, the implantation of intraocular lenses, corneal transplants, glaucoma filtration, retinal attachment and in the treatment of dry eyes. Finally, sodium hyaluronate is also used to coat the bladder lining in treating interstitial cystitis. Hyaluronan is similar to a substance that occurs naturally in the joints. It may work by acting as a lubricant and shock absorber in the joint, helping the knee to move smoothly, thereby lessening pain.

Rhamnan sulfate is a naturally occurring rhamnose-containing sulfated polysaccharide with antioxidant, antitcoagulant and antiviral biological activities. It is extracted from marine red, green and brown seaweeds (Patel, Therapeutic importance of sulfated polysaccharides from seaweeds: updating the recent findings. 3 Biotech, 2012, 2:171-185; Harada and Maeda, Chemical structure of antithrombin-active rhamnan sulfate from Monostrom nitidum. Bioscience, Biotechnology, and Biochemistry, 1998, 62:1647-1652). In certain embodiments, active agents for use with organophosphates include polysaccharides of plant, fungal or animal origin, such as plant- or fungal-derived pectins, galactomannans/mannoglycans, xyloglucans, and beta-glucans/lentinans. In certain embodiments, polysaccharides also include chitosan, alginate, fucoidan, galactan, carrageenan, k-carrageenan, galactofucan, mannoglucoronofucan, arabinogalactans, xylomannan sulfate, xylogalactofucan, dextran and derivatives thereof and ulvan.

Mannan from Candida albicans exhibits certain immunomodulatory properties. In general, these compounds consist mostly of a polysaccharide component but also include proteins (5% by weight). Mannose-binding lectins present on macrophages can bind mannan and activate the host immune system via a nonself-recognition mechanism (Tzianabos, Clinical Microbiology Reviews, 2000, 523-533).

Xyloglucans are polysaccharides that occur in the primary cell walls of all angiosperms (flowering plants) and medicinal mushrooms. Xyloglycans isolated from medicinal mushrooms exhibit antitumor effects (Wasser et al., Critical Reviews in Immunology, 1999, 19:65-96).

Alginate exhibits high capacity for water absorption and is capable of absorbing 200-300 times its own weight in water. Alginate is a linear copolymer with homopolymeric blocks of mannuronate and guluronate residues. Alginate is used in various pharmaceutical preparations as an inactive ingredient, such as in Gaviscon, Bisodol, and Asilone. Alginate is used extensively as an impression-making material in dentistry, prosthetics, lifecasting and occasionally for creating positives for small-scale casting. Alginate is also used as an additive in dehydrated products for slimming aids, and is used by the weight loss industry as an appetite suppressant.

Chitosan is a nontoxic and biodegradable polymer that has antibacterial, antiviral, antacid properties. Chitosan can also be used for film or fiber formation, or for forming hydrogels. (Chattopadhyay et al., International Journal of Polymer Science, 2010, 2010:1-7).

Fucoidan has been noted for antioxidant, immunostimulatory, lipid lowering, antibacterial and antihyperpeisic effects. Fucoidan and ulvan are also used in nanomedicine for wound healing, and for in vitro and in vivo controlled drug release (Patel, 3 Biotech, 2012, 2:171-185).

Galactan, carrageenan and k-carrageenan exhibit antioxidant, immunostimulatory, anti-inflammatory and antinociceptive, anticoagulant and antiviral effects. Galactofucan and mannoglucoronofucan may have antitumor effects. Arabinogalactants may have anticoagulant and antithrombotic effects. Xylomannan sulfate and xylogalactofucan exhibit antiviral effects, particularly against such viruses as influenza, herpes and human immunodeficiency virus (Patel, 3 Biotech, 2012, 2:171-185).

Dextran is a branched polysaccharide. Both dextran and many of its naturally-occurring and synthetic derivatives exhibit antithrombic activity. Additional polymers include, but are not limited to poly(ethylene glycol) based polymers; poly[(oligo ethyleneglycol) methacrylate] based polymers

### 2. Crosslinkers

Microgels can be formed of crosslinking the pNIPAm or polyketal polymers. Suitable crosslinkers include chemical univalent or multivalent crosslinkers. In preferred embodiments, the crosslinking agent for forming microgels is N,N′-methylenebisacrylamide (BIS). Other crosslinkers that may be used for forming microgels include, but are not limited to, 5-azido-2-nitrobenzoic acid N-hydroxysuccinimide ester, 4-azidophenacyl bromide, Benzophenone-4-isothiocyanate, 4-benzoylbenzoic acid N-succinimidyl ester, bis[2-(4-azidosalicylamido)ethyl] disulfide, 1,4-bis[3-(2-pyridyldithio)propionamido]butane, bis[2-(N-succinimidyl-oxycarbonyloxy)ethyl] sulfone, bromoacetic acid N-hydroxysuccinimide ester, 1,11-diazido-3,6,9-trioxaundecane, di(N-succinimidyl) glutarate, 3,3′-dithiodipropionic acid di(N-hydroxysuccinimide ester), iodoacetic acid N-hydroxysuccinimide ester, maleimidoacetic acid N-hydroxysuccinimide ester, 3-maleimidobenzoic acid N-hydroxysuccinimide ester, poly(ethylene glycol)-diacrylate, 4-(N-maleimido)benzophenone, and the like.

Optionally, the polymers forming microgels may be allowed to interact in a “crosslinker free” environment, forming ultra-low crosslinked (ULC) microgels via side reactions such as chain transfer.

### a. N,N′-methylenebisacrylamide (BIS)

In preferred embodiments, the crosslinking agent for forming microgels is N,N′-methylenebisacrylamide (BIS). Microgel crosslinking profoundly modulates individual microgel mechanical properties. Below their lower critical solution temperature (LCST), pNIPAm microgels crosslinked with 2 mol % BIS were found to have a Young's modulus of approximately 80 kPa, compared to 10 mol % BIS crosslinked particles, which were found to have a Young's modulus of approximately 500 kPa (Bachman et al., Soft Matter, 11:2018-2028 (2015)).

### b. Ultra-Low Crosslinked Microgels

Ultra-low crosslinked (ULC) pNIPAm microgels could be synthesized under ‘crosslinker free’ conditions. These particles are self-crosslinked through a chain transfer reaction at the teat-carbon sites which could occur on either the pendent isopropyl group or on the main chain backbone. These chain transfer reactions are rare, and therefore these particles have extremely low (<0.5%) degrees of crosslinking Previous studies have demonstrated that size and solid density of self-crosslinked pNIPAm microgels can be finely tuned by controlling reaction temperatures, monomer and initiator concentrations. Introduction of comonomers to these ULC microgels was shown to affect particle size, solid density and volume phase transition temperatures. Furthermore, hydrophobic comonomers, such as styrene and methyl methacrylate, were found to decrease particle size and increase solid density, while hydrophilic comonomers, such as acrylic acid (AAc), increased particle size and decreased solid density (Gao and Frisken, Langmuir, 19:5212-5216 (2003); Bachman et al., Soft Matter, 11:2018-2028 (2015)).

### 3. Shape and Size

In the fully formed colloidal assembly, microgels can have any shape. The shape of microgels may also change dependent on the density of packing of the microgels within the polymeric network, the density of the polymeric network, on the status of hydration of the colloidal assembly, and on the presence or absence of the infiltrating cells. Generally, the microgels are spherical in shape. The shape of the ULC microgels is more readily changeable by the above-listed factors, than the chemically-crosslinked microgels. In preferred embodiments, the hydrated microgels in the fully formed colloidal assembly are spherical in shape.

Typically, the diameter of the microgels ranges from 100 nanometers (nm) to 10 micrometers (μm). Preferably, the microgels have a diameter ranging from 500 nm to 5 μm.

C. Cells and Agents To Be Delivered

### 1. Cells

Examples of cells that may be incorporated into the compositions include, but are not limited to, embryonic stem cells, mesenchymal stem cells, induced pluripotent cells, totipotent cells, pluripotent or multipotent stem cells, adult stem cells, smooth muscle cells, endodermal cells, organ cells, skin cells, adipose cells, cardiac progenitor cells, cardiomyocytes, osteoclasts and osteoblasts, epithelial cells, and/or immune cells (T cells, B cells, monocytes, dendritic cells, neutrophils, eosinophils, and natural killer cells) optionally primed for detecting and engaging with endogenous cells, such as cancerous, infected, apoptotic or necrotic cells. The cells can be autologous or heterologous. Preferably, the cells are human. The cells can be genetically engineered to express a protein or secrete a protein or substance including, but not limited to growth factors and chemokines.

### 2. Active Agents

Agents to be delivered include therapeutic, nutritional, diagnostic, and prophylactic compounds. Proteins, peptides, carbohydrates, polysaccharides, nucleic acid molecules, and organic molecules, as well as diagnostic agents, can be delivered. The preferred materials to be incorporated are drugs and imaging agents. Therapeutic agents include antibiotics, antivirals, anti-parasites (helminths, protozoans), anti-cancer (referred to herein as “chemotherapeutics”, including cytotoxic drugs such as doxorubicin, cyclosporine, mitomycin C, cisplatin and carboplatin, BCNU, 5FU, methotrexate, adriamycin, camptothecin, epothilones A-F, and taxol), antibodies and bioactive fragments thereof (including humanized, single chain, and chimeric antibodies), antigen and vaccine formulations, peptide drugs, anti-inflammatories, nutraceuticals such as vitamins, and oligonucleotide drugs (including DNA, RNAs, antisense, aptamers, ribozymes, external guide sequences for ribonuclease P, and triplex forming agents).

Particularly preferred drugs to be delivered include growth-promoting hormones, cytokines, and tissue regenerative compounds.

Representative classes of diagnostic materials include paramagnetic molecules, fluorescent compounds, magnetic molecules, and radionuclides. Exemplary materials include, but are not limited to, metal oxides, such as iron oxide, metallic particles, such as gold particles, etc. Biomarkers can also be incorporated for diagnostic applications.

Active agents include therapeutic, prophylactic, neutraceutical and diagnostic agents. Any suitable agent may be used. These include organic compounds, inorganic compounds, proteins, polysaccharides, nucleic acids or other materials that can be incorporated using standard techniques.

Active agents include synthetic and natural proteins (including enzymes, peptide-hormones, receptors, growth factors, antibodies, signaling molecules), and synthetic and natural nucleic acids (including RNA, DNA, anti-sense RNA, triplex DNA, inhibitory RNA (RNAi), and oligonucleotides), and biologically active portions thereof. Suitable active agents have a size greater than about 1,000 Da for small peptides and polypeptides, more typically at least about 5,000 Da and often 10,000 Da or more for proteins. Nucleic acids are more typically listed in terms of base pairs or bases (collectively “bp”). Nucleic acids with lengths above about 10 by are typically incorporated. More typically, useful lengths of nucleic acids for probing or therapeutic use will be in the range from about 20 by (probes; inhibitory RNAs, etc.) to tens of thousands of by for genes and vectors. The active agents may also be hydrophilic molecules, preferably having a low molecular weight.

Examples of useful proteins include hormones such as insulin and growth hormones including somatomedins. Examples of useful drugs include neurotransmitters such as L-DOPA, antihypertensives or saluretics such as Metolazone from Searle Pharmaceuticals, carbonic anhydrase inhibitors such as Acetazolamide from Lederle Pharmaceuticals, insulin like drugs such as glyburide, a blood glucose lowering drug of the sulfonylurea class, synthetic hormones such as Android F from Brown Pharmaceuticals and Testred® (methyltestosterone) from ICN Pharmaceuticals.

Representative anti-cancer agents include, but are not limited to, alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel, epothilones A-F, and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), and combinations thereof. Other suitable anti-cancer agents include angiogenesis inhibitors including antibodies to vascular endothelial growth factor (VEGF) such as bevacizumab (AVASTIN®), other anti-VEGF compounds; thalidomide (THALOMID®) and derivatives thereof such as lenalidomide (REVLIMID®); endostatin; angiostatin; receptor tyrosine kinase (RTK) inhibitors such as sunitinib (SUTENT®); tyrosine kinase inhibitors such as sorafenib (Nexavar®), erlotinib (Tarceva®), pazopanib, axitinib, and lapatinib; transforming growth factor-a or transforming growth factor-β inhibitors, and antibodies to the epidermal growth factor receptor such as panitumumab (VECTIBIX®) and cetuximab (ERBITUX®).

Under the Biopharmaceutical Classification System (BCS), drugs can belong to four classes: class I (high permeability, high solubility), class II (high permeability, low solubility), class III (low permeability, high solubility) or class IV (low permeability, low solubility). Suitable active agents also include poorly soluble compounds; such as drugs that are classified as class II or class IV compounds using the BCS. Examples of class II compounds include: acyclovir, nifedipine, danazol, ketoconazole, mefenamic acid, nisoldipine, nicardipine, felodipine, atovaquone, griseofulvin, troglitazone glibenclamide and carbamazepine. Examples of class IV compounds include: chlorothiazide, furosemide, tobramycin, cefuroxmine, and paclitaxel.

For imaging, radioactive materials such as Technetium99 (^{99m}Tc) or magnetic materials such as Fe₂O₃ could be used.

Alternatively, the compositions may encapsulate cellular materials, such as for example, cellular materials to be delivered to antigen presenting cells as described below to induce immunological responses.

Dendritic cells (DCs) are recognized to be powerful antigen presenting cells for inducing cellular immunologic responses in humans. DCs prime both CD8 + cytotoxic T-cell (CTL) and CD4+ T-helper (Th1) responses. DCs are capable of capturing and processing antigens, and migrating to the regional lymph nodes to present the captured antigens and induce T-cell responses. Immature DCs can internalize and process cellular materials, such as DNA encoding antigens, and induce cellular immunologic responses to disease effectors.

### 3. Nucleic Acids

Any nucleic acid for therapeutic, diagnostic, clinical or drug delivery use (collectively referred to herein as functional nucleic acids) can be delivered with the colloidal assemblies. Functional nucleic acids can be divided into the following non-limiting categories: copyDNA (cDNA), DNA aptamers, DNAzymes, RNA aptamers, external guide sequences, RNA interference molecules, such as small interfering RNA, antisense RNA, short hairpin RNA, and micro RNA (miRNA), morpholinos, messenger RNA (mRNA), long non-coding RNA (lincRNA), as well as ribozymes, triplex-forming molecules and nucleic acid containing nanoparticles. Therapeutic nucleic acids are capable of modulating functionality of the genes once they arrive within a cell. Introduction of foreign nucleic acid into a cell can be accomplished by viral transduction and non-viral delivery, such as ultrasound, electroporation, lipid-dependent delivery, polypeptide-dependent delivery, calcium co-precipitation, transfection with a “naked” nucleic acid molecule, self-delivering nucleic acid conjugates, and polymer- or glycopolymer-dependent (i.e., polyethyleneimine) delivery in some cases utilizing nanomaterials to shape nucleic acid-containing nano- and micro-particles.

### a. Functional Nucleic Acids

### i. Antisense

The functional nucleic acids can be antisense molecules. Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively, the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. It is preferred that antisense molecules bind the target molecule with a dissociation constant (Kd) less than or equal to 10⁻⁶, 10⁻⁸, 10⁻¹⁰ or 10⁻¹².

### ii. Aptamers

The functional nucleic acids can be aptamers. Aptamers are DNA or RNA molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with Kd's from the target molecule of less than 10⁻¹² M. It is preferred that the aptamers bind the target molecule with a Kd less than 10⁻⁶, 10⁻⁸, 10⁻¹⁰, or 10⁻¹². Aptamers can bind the target molecule with a very high degree of specificity. For example, aptamers have been isolated that have greater than a 10,000-fold difference in binding affinities between the target molecule and another molecule that differ at only a single position on the molecule. It is preferred that the aptamer have a Kd with the target molecule at least 10-, 100-, 1,000-, 10,000-, or 100,000-fold lower than the Kd with a background binding molecule.

### iii. Ribozymes

The functional nucleic acids can be ribozymes. Ribozymes are RNA molecules that are capable of catalyzing a chemical reaction, either intramolecularly or intermolecularly. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substiate is based on the target substrates sequence.

### iv. Triplex Forming Oligonucleotides

The functional nucleic acids can be triplex forming molecules. Triplex forming functional nucleic acid molecules are molecules that can interact with either a double-stranded or single-stranded nucleic acid. When triplex molecules interact with a target region, a structure called a triplex is formed in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a Kd less than 10⁻⁶, 10⁻⁸, 10⁻¹⁰, or 10⁻¹².

### v. External Guide Sequences

The functional nucleic acids can be external guide sequences. External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukaryotic cells. Representative examples of how to make and use EGS molecules to facilitate cleavage of a variety of different target molecules are known in the art.

### vi. RNA Interference

In some embodiments, the functional nucleic acids induce gene silencing through RNA interference. Gene expression can also be effectively silenced in a highly specific manner through RNA interference (RNAi). This silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III-like enzyme, Dicer, into double stranded small interfering RNAs (siRNA) 21-23 nucleotides in length that contain 2 nucleotide overhangs on the 3′ ends (Elbashir, et al. (2001) Genes Dev., 15:188-200; Bernstein, et al. (2001) Nature, 409:363-6; Hammond, et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, et al. (2001) Cell, 107:309-21). At some point the siRNA duplex unwinds, and the antisense strand remains bound to RISC which directs degradation of the complementary mRNA sequence by a combination of endo and exonucleases (Martinez, et al. (2002) Cell, 110:563-74). However, the effect of RNAi or siRNA or their use is not limited to any type of mechanism.

Small Interfering RNA (siRNA) is a double-stranded RNA that can induce sequence-specific post-transcriptional gene silencing, thereby decreasing or even inhibiting gene expression. In one example, a siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends.

Sequence specific gene silencing can be achieved in mammalian cells using synthetic, short double-stranded RNAs that mimic the siRNAs produced by the enzyme Dicer (Elbashir, et al. (2001) Nature, 411:494 498) (Ui-Tei, et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAs (shRNAs). Kits for the production of vectors containing shRNA are available, such as, for example, Imgenex's GENESUPPRESSOR™ Construction Kits and Invitrogen's BLOCK-IT™ inducible RNAi plasmid and lentivirus vectors.

In some embodiments, the functional nucleic acid is siRNA, shRNA, or micro RNA (miRNA). In some embodiments, the composition includes a vector expressing the functional nucleic acid. Methods of making and using vectors for in vivo expression of functional nucleic acids such as antisense oligonucleotides, siRNA, shRNA, miRNA, EGSs, ribozymes, and aptamers are known in the art.

### vii. Other Gene Editing Compositions

In some embodiments the functional nucleic acids are gene editing compositions. Gene editing compositions can include nucleic acids that encode an element or elements that induce a single or a double strand break in the target cell's genome, and optionally a polynucleotide.

### b. Strand Break Inducing Elements

### i. CRISPR/Cas

In some embodiments, the element that induces a single or a double strand break in the target cell's genome is a CRISPR/Cas system. CRISPR (Clustered Regularly Interspaced Short Palindromic Repeats) is an acronym for DNA loci that contain multiple, short, direct repetitions of base sequences. The prokaryotic CRISPR/Cas system has been adapted for gene editing use (silencing, enhancing or changing specific genes) in eukaryotes (see, for example, Cong, Science, 15:339(6121):819-823 (2013) and Jinek, et al., Science, 337(6096):816-21 (2012)). By transfecting a cell with the required elements including a cas gene and specifically designed CRISPRs, the organism's genome can be cut and modified at any desired location. Methods of preparing compositions for use in genome editing using the CRISPR/Cas systems are described in detail in WO 2013/176772 and WO 2014/018423.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease.

In some embodiments, a tracrRNA and crRNA are linked and form a chimeric crRNA-tracrANA hybrid where a mature crRNA is fused to a partial tracrRNA via a synthetic stem loop to mimic the natural crRNA:tracrRNA duplex as described in Cong, Science, 15:339(6121):819-823 (2013) and Jinek, et al., Science, 337(6096):816-21 (2012). A single fused crRNA-tracrRNA construct can also be referred to as a guide RNA or gRNA (or single-guide RNA (sgRNA)). Within a sgRNA, the crRNA portion can be identified as the ‘target sequence’ and the tracrRNA is often referred to as the ‘scaffold’.

In some embodiments, one or more vectors driving expression of one or more elements of a CRISPR system are introduced into a target cell such that expression of the elements of the CRISPR system direct formation of a CRISPR complex at one or more target sites. While the specifics can be varied in different engineered CRISPR systems, the overall methodology is similar. A practitioner interested in using CRISPR technology to target a DNA sequence can insert a short DNA fragment containing the target sequence into a guide RNA expression plasmid. The sgRNA expression plasmid contains the target sequence (about 20 nucleotides), a form of the tracrRNA sequence (the scaffold), as well as a suitable promoter and necessary elements for proper processing in eukaryotic cells. Such vectors are commercially available (see, for example, Addgene). Many of the systems rely on custom, complementary oligos that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site.

### ii. Zinc Finger Nucleases

In some embodiments, the nucleic acid construct or constructs encoding zinc finger nucleases (ZFNs). ZFNs are typically fusion proteins that include a DNA-binding domain derived from a zinc-finger protein linked to a cleavage domain

The most common cleavage domain is the Type IIS enzyme Fokl. Fokl catalyzes double-stranded cleavage of DNA, at 9 nucleotides from its recognition site on one strand and 13 nucleotides from its recognition site on the other. See, for example, U.S. Pat. Nos. 5,356,802; 5,436, 150 and 5,487,994; as well as Li et al. Proc., Natl. Acad. Sci. USA 89 (1992):4275-4279; Li et al. Proc. Natl. Acad. Sci. USA, 90:2764-2768 (1993); Kim et al. Proc. Natl. Acad. Sci. USA. 91:883-887 (1994a); Kim et al. J. Biol. Chem. 269:31, 978-31,982 (1994b). One or more of these enzymes (or enzymatically functional fragments thereof) can be used as a source of cleavage domains.

The DNA-binding domain, which can, in principle, be designed to target any genomic location of interest, can be a tandem array of Cys2His2 zinc fingers, each of which generally recognizes three to four nucleotides in the target DNA sequence. The Cys2His2 domain has a general structure: Phe (sometimes Tyr)-Cys-(2 to 4 amino acids)-Cys-(3 amino acids)-Phe(sometimes Tyr)-(5 amino acids)-Leu-(2 amino acids)-His-(3 amino acids)-His. By linking together multiple fingers (the number varies: three to six fingers have been used per monomer in published studies), ZFN pairs can be designed to bind to genomic sequences 18-36 nucleotides long.

### iii. Transcription Activator-Like Effector Nucleases

In some embodiments, nucleic acid construct(s) encode a transcription activator-like effector nuclease (TALEN). TALENs have an overall architecture similar to that of ZFNs, with the main difference that the DNA-binding domain comes from TAL effector proteins, transcription factors from plant pathogenic bacteria. The DNA-binding domain of a TALEN is a tandem array of amino acid repeats, each about 34 residues long. The repeats are very similar to each other; typically they differ principally at two positions (amino acids 12 and 13, called the repeat variable diresidue, or RVD). Each RVD recognizes a specific nucleotide, leading to a simple code for DNA recognition: NI for adenine, HD for cytosine, NG for thymine and NH or NN for guanine. Each RVD specifies preferential binding to one of the four possible nucleotides, meaning that each TALEN repeat binds to a single base pair, though the NN RVD is known to bind adenines in addition to guanine TAL effector DNA binding is mechanistically less well understood than that of zinc-finger proteins, but their seemingly simpler code could prove very beneficial for engineered-nuclease design. TALENs also cleave as dimers, have relatively long target sequences (the shortest reported so far binds 13 nucleotides per monomer) and appear to have less stringent requirements than ZFNs for the length of the spacer between binding sites. Monomeric and dimeric TALENs can include more than 10, more than 14, more than 20, or more than 24 repeats.

Methods of engineering TAL to bind to specific nucleic acids are described in Cermak, et al, Nucl. Acids Res. 1-11 (2011); US Published Application No. 2011/0145940, which discloses TAL effectors and methods of using them to modify DNA. Miller et al. Nature Biotechnol 29: 143 (2011) reported making TALENs for site-specific nuclease architecture by linking TAL truncation variants to the catalytic domain of Fold nuclease. The resulting TALENs were shown to induce gene modification in immortalized human cells. General design principles for TALEN binding domains can be found in, for example, WO 2011/072246.

### c. Gene Altering Polynucleotides

The nuclease activity of the genome editing systems cleave target DNA to produce single or double strand breaks in the target DNA. Double strand breaks can be repaired by the cell in one of two ways: non-homologous end joining, and homology-directed repair. In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence is used as a template for repair of the cleaved target DNA sequence, resulting in the transfer of genetic information from a donor polynucleotide to the target DNA. As such, new nucleic acid material can be inserted/copied into the site.

Therefore, in some embodiments, the genome editing composition optionally includes a donor polynucleotide. The modifications of the target DNA due to NHEJ and/or homology-directed repair can be used to induce gene correction, gene replacement, gene tagging, transgene insertion, nucleotide deletion, gene disruption, gene mutation, etc.

Accordingly, cleavage of DNA by the genome editing composition can be used to delete nucleic acid material from a target DNA sequence by cleaving the target DNA sequence and allowing the cell to repair the sequence in the absence of an exogenously provided donor polynucleotide. Alternatively, if the genome editing composition includes a donor polynucleotide sequence that includes at least a segment with homology to the target DNA sequence, the methods can be used to add, i.e., insert or replace, nucleic acid material to a target DNA sequence (e.g., to “knock in” a nucleic acid that encodes for a protein, an siRNA, an miRNA, etc.), to add a tag (e.g., 6xHis, a fluorescent protein (e.g., a green fluorescent protein; a yellow fluorescent protein, etc.), hemagglutinin (HA), FLAG, etc.), to add a regulatory sequence to a gene (e.g., promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, the compositions can be used to modify DNA in a site-specific, i.e., “targeted”, way, for example gene knock-out, gene knock-in, gene editing, gene tagging, etc. as used in, for example, gene therapy.

In applications in which it is desirable to insert a polynucleotide sequence into a target DNA sequence, a polynucleotide including a donor sequence to be inserted is also provided to the cell. By a “donor sequence” or “donor polynucleotide” or “donor oligonucleotide” it is meant a nucleic acid sequence to be inserted at the cleavage site. The donor polynucleotide typically contains sufficient homology to a genomic sequence at the cleavage site, e.g., 70%, 80%, 85%, 90%, 95%, or 100% homology with the nucleotide sequences flanking the cleavage site, e.g., within about 50 bases or less of the cleavage site, e.g., within about 30 bases, within about 15 bases, within about 10 bases, within about 5 bases, or immediately flanking the cleavage site, to support homology-directed repair between it and the genomic sequence to which it bears homology. The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence includes a non-homologous sequence flanked by two regions of homology, such that homology-directed repair between the target DNA region and the two flanking sequences results in insertion of the non-homologous sequence at the target region.

### d. Oligonucleotide Composition

The functional nucleic acids can be DNA or RNA nucleotides which typically include a heterocyclic base (nucleic acid base), a sugar moiety attached to the heterocyclic base, and a phosphate moiety which esterifles a hydroxyl function of the sugar moiety. The principal naturally-occurring nucleotides comprise uracil, thymine, cytosine, adenine and guanine as the heterocyclic bases, and ribose or deoxyribose sugar linked by phosphodiester bonds.

In some embodiments, the oligonucleotides are composed of nucleotide analogs that have been chemically modified to improve stability, half-life, or specificity or affinity for a target receptor, relative to a DNA or RNA counterpart. The chemical modifications include chemical modification of nucleobases, sugar moieties, nucleotide linkages, or combinations thereof. As used herein “modified nucleotide” or “chemically modified nucleotide” defines a nucleotide that has a chemical modification of one or more of the heterocyclic base, sugar moiety or phosphate moiety constituents. In some embodiments, the charge of the modified nucleotide is reduced compared to DNA or RNA oligonucleotides of the same nucleobase sequence. For example, the oligonucleotide can have low negative charge, no charge, or positive charge.

Typically, nucleoside analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). In some embodiments, the analogs have a substantially uncharged, phosphorus containing backbone.

### i. Locked Nucleic Acids

In another embodiment, the oligonucleotides are composed of locked nucleic acids. Locked nucleic acids (LNA) are modified RNA nucleotides (see, for example, Braasch, et al., Chem. Biol., 8(1):1-7 (2001)). LNAs form hybrids with DNA which are more stable than DNA/DNA hybrids, a property similar to that of peptide nucleic acid (PNA)/DNA hybrids. Therefore, LNA can be used just as PNA molecules would be. LNA binding efficiency can be increased in some embodiments by adding positive charges to it. Commercial nucleic acid synthesizers and standard phosphoramidite chemistry are used to make LNAs.

### ii. Peptide Nucleic Acids

In some embodiments, the oligonucleotides are composed of peptide nucleic acids. Peptide nucleic acids (PNAs) are synthetic DNA mimics in which the phosphate backbone of the oligonucleotide is replaced in its entirety by repeating N-(2-aminoethyl)-glycine units and phosphodiester bonds are typically replaced by peptide bonds. The various heterocyclic bases are linked to the backbone by methylene carbonyl bonds. PNAs maintain spacing of heterocyclic bases that is similar to conventional DNA oligonucleotides, but are achiral and neutrally charged molecules. Peptide nucleic acids are comprised of peptide nucleic acid monomers.

Other backbone modifications include peptide and amino acid variations and modifications. Thus, the backbone constituents of oligonucleotides such as PNA may be naturally occurring and non-naturally occurring peptide linkages. Examples of non-naturally occurring peptide linkages include those in which one or more nitrogen atoms is(are) acetylated, or linkages may include amino spacers such as 8-amino-3,6-dioxaoctanoic acid. Amino acids residues such as lysine are particularly useful if positive charges are desired in the PNA. Methods for the chemical assembly of PNAs are well known. See, for example, U.S. Pat. Nos. 5,539,082, 5,527,675, 5,623,049, 5,714,331, 5,736,336, 5,773,571 and 5,786,571.

### iii. Heterocyclic Bases

The principal naturally-occurring nucleotides include uracil, thymine, cytosine, adenine and guanine as the heterocyclic bases. The oligonucleotides can include chemical modifications to their nucleobase constituents. Chemical modifications of heterocyclic bases or heterocyclic base analogs may be effective to increase the binding affinity or stability in binding a target sequence. Chemically-modified heterocyclic bases include, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives.

### iv. Sugar Modifications

Oligonucleotides can also contain nucleotides with modified sugar moieties or sugar moiety analogs. Sugar moiety modifications include, but are not limited to, 2′-O-aminoethoxy, 2′-O-aminoethyl (2′-OAE), 2′-O-methoxy, 2′-O-methyl, 2-guanidoethyl (2′-OGE), 2′-O,4′-C-methylene (LNA), 2′-O-(methoxyethyl) (2′-OME) and 2′-O-(N-(methyl)acetamido) (2′-OMA).

### v. Morpholinos

In some embodiments, the functional nucleic acid is a morpholino oligonucleotide. Morpholino oligonucleotides are typically composed of two or more morpholino monomers containing purine or pyrimidine base-pairing moieties effective to bind, by base-specific hydrogen bonding, to a base in a polynucleotide, which are linked together by phosphorus-containing linkages, one to three atoms long, joining the morpholino nitrogen of one monomer to the 5′-exocyclic carbon of an adjacent monomer. The purine or pyrimidine base-pairing moiety is typically adenine, cytosine, guanine, uracil or thymine The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337.

Important properties of the morpholino-based subunits typically include: the ability to be linked in an oligomeric form by stable, uncharged backbone linkages; the ability to support a nucleotide base (e.g. adenine, cytosine, guanine, thymidine, uracil or inosine) such that the polymer formed can hybridize with a complementary-base target nucleic acid, including target RNA, with high melting temperature, even with oligomers as short as 10-14 bases; the ability of the oligomer to be actively transported into mammalian cells; and the ability of an oligomer:RNA heteroduplex to resist RNAse degradation.

In some embodiments, oligonucleotides employ morpholino-based subunits bearing base-pairing moieties, joined by uncharged linkages, as described above.

### vi. Internucleotide Linkages

Modifications to the phosphate backbone of a DNA or RNA oligonucleotide may increase the binding affmity or stability of the oligonucleotide, or reduce the susceptibility of oligonucleotides to nuclease digestion. Cationic modifications, including, but not limited to, diethyl-ethylenediamide (DEED) or dimethyl-aminopropylamine (DMAPA) may be especially useful due to decreased electrostatic repulsion between the oligonucleotide and a target. Modifications of the phosphate backbone may also include the substitution of a sulfur atom for one of the non-bridging oxygens in the phosphodiester linkage. This substitution creates a phosphorothioate internucleoside linkage in place of the phosphodiester linkage. Oligonucleotides containing phosphorothioate internucleoside linkages have been shown to be more stable to nucleases in vivo.

Examples of modified nucleotides with reduced charge include modified internucleotide linkages such as phosphate analogs having achiral and uncharged intersubunit linkages (e.g., Sterchak, E. P. et al., Organic Chem., 52:4202, (1987)), and uncharged morpholino-based polymers having achiral intersubunit linkages (see, e.g., U.S. Pat. No. 5,034,506), as discussed above. Some internucleotide linkage analogs include morpholidate, acetal, and polyamide-linked heterocycles.

### vii. Terminal Residues

Oligonucleotides optionally include one or more terminal residues or modifications at either or both termini to increase stability, and/or affinity of the oligonucleotide for its target. Commonly used positively-charged moieties include the amino acids lysine and arginine, although other positively-charged moieties may also be useful. Oligonucleotides may further be modified to be end capped to prevent degradation using a propylamine group. Procedures for 3′ or 5′ capping oligonucleotides are well known in the art.

In some embodiments, the functional nucleic acid can be single stranded or double stranded.

D. Factors Promoting Cell Infiltration

### 1. Cytokines

Exemplary cytokines that may be incorporated into the matrix-microgel composites include, but are not limited to, members of the interleukin (IL)-2 subfamily cytokines, the interferons (IFN)-α, IFN-β, and IFN-γ, the IL-10 subfamily cytokines, IL-1, IL-4, IL-6, IL-10, IL-12, IL-13, IL-17, IL-18, tumor necrosis factor (TNF)-α, transforming growth factor (TGF)-β1, TGF-β2 and TGF-β3, VEGF, EGF, IGF, FGF, and BMP-2.

### 2. Chemokines

Exemplary chemokines that may be incorporated into the colloidal assemblies include, but are not limited to, homeostatic chemokines CCL14, CCL19, CCL20, CCL21, CCL25, CCL27, CXCL12 (SDF-1) and CXCL13, and inflammatory chemokines CXCL-8 (IL-8), CCL2, CCL3, CCL4, CCL5 (RANTES), CCL11, and CXCL10. In preferred embodiments, the chemokine is SDF-1.

### 3. Protease and Protease Inhibitors

Protease and protease inhibitors can also be loaded into or on the disclosed compositions. In one embodiment, the proteases and/or protease inhibitors are added to modulated porosity of the compositions to promote or reduce cell penetration in to the compositions. The modulation of porosity can occur over time, for example after the composition has been administered to a subject

### Proteases

Proteases are can generally by classified into six broad groups: serine proteases (using a serine alcohol), threonine proteases (using a threonine secondary alcohol), cysteine proteases (using a cysteine thiol), aspartate proteases (using an aspartate carboxylic acid), glutamic acid proteases (using a glutamate carboxylic acid), metalloproteases (using a metal, usually zinc).

Exemplary serine proteases include, but are not limited to, chymotrypsin A (Bos taurus), penicillin G acylase precursor (Escherichia coli), dipeptidase E (Escherichia coli), DmpA aminopeptidase (Ochrobactrum anthropi), subtilisin (Bacillus licheniformis), prolyl oligopeptidase (Sus scrofa), D-Ala-D-Ala peptidase C (Escherichia coli), signal peptidase I (Escherichia coli), cytomegalovirus assemblin (human herpesvirus 5), Lon-A peptidase (Escherichia coli), peptidase Clp (Escherichia coli), Escherichia coli phage K1F endosialidase CIMCD self-cleaving protein (Enterobacteria phage K1F), nucleoporin 145 (Homo sapiens), lactoferrin (Homo sapiens), murein tetrapeptidase LD-carboxypeptidase (Pseudomonas aeruginosa), and rhomboid-1 (Drosophila melanogaster)

Exemplary threonine proteases include, but are not limited to, archaean proteasome, beta component (Thermoplasma acidophilum), and ornithine acetyltransferase (Saccharomyces cerevisiae).

Exemplary cysteine proteases include, but are not limited to, TEV protease (Tobacco etch virus), amidophosphoribosyltransferase precursor (Homo sapiens), gamma-glutamyl hydrolase (Rattus norvegicus), hedgehog protein (Drosophila melanogaster), DmpA aminopeptidase (Ochrobactrum anthropi), papain (Carica papaya), bromelain (Ananas comosus), cathepsin K (liverwort), calpain (Homo sapiens), caspase-1 (Rattus norvegicus), separase(Saccharomyces cerevisiae), adenain (human adenovirus type 2), pyroglutamyl-peptidase I (Bacillus amyloliquefaciens), sortase A (Staphylococcus aureus), hepatitis C virus peptidase 2 (hepatitis C virus), sindbis virus-type nsP2 peptidase (sindbis virus), dipeptidyl-peptidase VI (Lysinibacillus sphaericus), and DeSI-1 peptidase (Mus musculus).

Exemplary aspartate proteases include, but are not limited to, BACE1, BACE2, cathepsin D, cathepsin E, chymosin (or “rennin”), napsin-A, nepenthesin, pepsin, plasmepsin, presenilin, and renin

Exemplary metalloproteases include, but are not limited to, regulatory element binding protein (SREBP) site 2 protease and Escherichia coli protease EcfE, stage IV sporulation protein FB.

### Protease Inhibitors

Common protease inhibitors used in research and development include, but are not limited to, inhibitors of serine proteases, cysteine proteases, calpains, and metalloproteinases, and economical alternatives thereof, such as 1,10-phenanthroline, AEBSF, antipain, aprotinin, bestatin, bestatin hydrochloride, chymostatin, E-64, EDTA, elastatinal, ε-aminocaproic acid, N-Ethylmaleimide, leupeptin, nafamostat mesylate, pepstatin A, phosphoramidon, phosphoramidon disodium salt, trypsin inhibitor, alpha 1-antitrypsin, C1-inhibitor, antithrombin, alpha 1-antichymotrypsin, plasminogen activator inhibitor-1, neuroserpin, and combinations and cocktails thereof.

Therapeutic protease inhibitors include, but are not limited to, amprenavir (AGENERASE®), atazanavir (REYATAZ®), darunavir (PREZISTA®), fosamprenavir (TELZIR®, LEXIVA®), indinavir (CRIXIVAN®), lopinavir/ritonavir (KALETRA®, ALUVIA®), nelfinavir (VIRACEPT®), ritonavir (NORVIR®), saquinavir (INVIRASE®), tipranavir (APTIVUS®), darunavir+cobicistat (PREZCOBIX®), atazanavir+cobicistat (EVOTAZ®)

### III. Methods of Making

A. Methods of Making Microgels

Ultralow crosslinked microgels may be made according to the procedures laid out in Gao and Frisken Influence of reaction conditions on the synthesis of self-crosslinked N-isopropylacrylamide microgels. Langmuir 19, 5217-5222 (2003) and Bachman et al. Soft Matter, 2015, 11, 2018, which are incorporated herein by reference in its entirety. As described in the Examples, in one embodiment a 95%pNIPAm/5% AAc composition (poly(N-isopropylacrylamide-co-acrylic acid) may be utilized for microgel synthesis which results in microscaffolds ˜1-μm diameter in the swollen state.

B. Methods of Making the Compositions.

In one embodiment, the compositions are formed of incorporating 1) polymers/monomers, 2) microgels, and 3) a chemical or physical polymerizing or cross-linking agent in an aqueous medium. The choice of the material that will form the polymer network governs the choice of the polymerizing or cross-linking agent and the nature of the aqueous medium. In preferred embodiments, the polymer network is formed of fibrin. In this embodiment, the fibrin and microgels are mixed in a buffer containing CaCl₂ NaCl, and buffered to pH of 7.4. The matrix-microgel composite is then formed by addition of thrombin at a concentration of between 0.1 and 10 U/ml to aid colloidal assembly formation.

### IV. Kits

Kits containing vials of different concentrations of the components for the ex vivo or in situ formation of the colloidal assemblies are provided.

A. Polymers, Microgels, and Polymerizing Agents.

The kits may contain a plurality of, such as two to 100, vials of monomers or polymers, microgels, and polymerizing or cross-linking agents in a container. Typically, the kits contain at least one vial of the monomer or polymer in wet or lyophilized form, at least one vial of microgels, in dry or swollen form, and at least one vial of a polymerizing or cross-linking agent, in wet or lyophilized form. Optionally, the kits may contain a plurality of vials with a combination of lyophilized or wet monomers or polymers and microgels. The plurality of vials may contain the same or different quantities of monomers or polymers, microgels, combinations thereof, or polymerizing or cross-linking agents.

B. Buffers

The kits may optionally contain vials, pouches, or containers with buffers. The buffers may be in powder form or in solution. When in solution, the buffers may be of various concentrations.

C. Instructions for Use

The kits may contain instructions for use. The instructions for use are typically tailored to the kit content, as some kits may require the addition of water only to a powdered buffer or to a concentrated buffer before combining with the monomer or polymer, microgel, and the polymerizing or cross-linking agent.

### V. Methods of Using the Compositions

A. Hemostasis

The disclosed compositions can be used to induce, promote or increase hemostasis in a subject, preferably a human. By increasing hemostasis in the subject, bleeding or bloods loss is reduced. One embodiment provides a method of promoting or increasing in subject in need thereof by administering to a subject in need thereof, an effective amount of the disclosed compositions that partially or fully fill the wound to reduce blood loss or bleeding or to promote or increase clot formation in the subject. The compositions can be administered parenterally or topically depending on whether the bleeding is internal or through the skin.

Another embodiment provides a method of promoting or increasing clot contraction in a subject in need thereof, by administering an effective amount of the composition to increase clot contraction in the subject relative to a control colloidal assemblies. Controls can be compositions that do not contain a microgels.

The compositions can be administered to any type of wounds to promote hemostasis. The wound can be internal or external. For example, the wound can be caused by a weapon such as a gun, knife, or explosive or as a result of an accident such as an automobile accident.

B. Fibrotic and Chronic Wounds

The disclosed compositions can be used to treat chronic wounds. A chronic wound is a wound that does not heal normally. Wounds that do not heal within three months are often considered chronic. One embodiment provides administering an effective amount of the colloidal assemblies to a chronic wound to promote or enhance hemostasis.

The disclosed colloidal assemblies can also be used to treat fibrotic wounds. Fibrotic wounds have dysregulated healing and typically delayed healing. Fibrosis can be defined as the replacement of the normal structural elements of the tissue by distorted, non-functional and excessive accumulation of scar tissue. Another embodiment provides a method for treating fibrotic wounds by administering an effective amount of the disclosed compositions to promote or enhance hemostasis, to promote or enhance infiltration of tissue regenerating cells into the compositions, and to prevent deposition of fibrotic scars.

C. Drug Delivery

In some embodiments, the compositions can contain, be loaded with or functionalized with a bioactive agent. The compositions can be used to deliver an effective amount of one or more therapeutic, diagnostic, and/or prophylactic agents to an individual in need of such treatment. The amount of agent to be administered can be readily determine by the prescribing physician and is dependent on the age and weight of the patient and the disease or disorder to be treated.

The compositions are useful in drug delivery (as used herein “drug” includes therapeutic, nutritional, diagnostic and prophylactic agents), whether injected or surgically administered. The compositions may be administered pre-formed or in an aqueous suspension (in water, saline, buffered saline, etc.) to form in situ.

The compositions can be used to as delivery vehicles for a number of active agent cargos including small molecules, nucleic acids, proteins, and other bioactive agents. The active agent or agents can be encapsulated within, dispersed within, and/or associated with the surface of the microgel. In some embodiments, the microgel packages two, three, four, or more different active agents for simultaneous delivery. In a preferred embodiment, the active agent is a cytokine or growth factor. Exemplary cytokines and growth factors include, but are not limited to members of the epidermal growth factor (EGF) family, transforming growth factor beta (TGF-beta) family, fibroblast growth factor (FGF) family, vascular endothelial growth factor (VEGF), granulocyte macrophage colony stimulating factor (GM-CSF), platelet-derived growth factor (PDGF), connective tissue growth factor (CTGF), interleukin (IL) family, and tumor necrosis factor-alpha family. Specific cytokines and growth factors include but are not limited to TGF-beta, EGF, TGF-alpha, VEGF, IGF-I, FGFs, IL-1beta, IL-4, IL-6, IL-8, IFN-alpha/beta, PDGF-BB, bFGF, and GM-CSF.

The active agent can be an anti-tumor agent or chemotherapeutic agent Representative chemotherapeutic agents include, but are not limited to amsacrine, bleomycin, busulfan, capecitabine, carboplatin,carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Representative pro-apoptotic agents include, but are not limited to fludarabinetaurosporine, cycloheximide, actinomycin D, lactosylceramide, 15d-PGJ(2) and combinations thereof.

The acellular or cellular colloidal assemblies, containing active agents, can be used to treat a variety of diseases and conditions, for example, tissue repair and regeneration, drug delivery to growing or repairing tissue, and sealing of acute and chronic wounds. The colloidal assemblies can be administered to the subject therapeutically or prophylactically.

D. Medical Devices

The disclosed compositions can be used to coat medical devices. Preferred medical devices that can be coated with the microgels or matrix microgel composites assemblies include, but are not limited to medical devices that are used to inhibit or reduce bleeding or blood loss. Exemplary medical devices that can be coated with the microgels or matrix-microgel composites include clamps, staples and sutures.

### 1. Wound Dressing

The compositions can also be used with wound dressings. One embodiment provides a wound dressing having a layer of the compositions on the wound dressing. The layer of polymeric matrix containing microgels is configured to come into contact with the wound when the wound dressing is applied to a wound. The compositions can be impregnated in the wound dressing or coated on the wound dressing using conventional techniques. The wound dressing can be made of absorbent materials such as cotton of fleece. The wound dressing can also be made of synthetic fibers for example polyamide fibers. In certain embodiments, the wound dressing can have multiple layers including an adhesive layer, an absorbent layer, and moisture regulation layer. The wound dressing can also include antimicrobial agents, antifungal agents, and other active agents to promote wound healing such as cytokines and growth factors discussed above.

### 2. Sealants

The disclosed compositions can be used as a sealant or tissue adhesive to seal ruptures or open wounds by promoting blood clotting or for anastomosis. The compositions can be formulated as a dry powder or aqueous suspension and packaged into discrete packets or units to form a kit. Surgical hemostatic agents and sealants may be used as an aid to cease hemorrhage during surgery, either mechanically or by augmenting the body's response to coagulation. Their application can extend to the cessation of bleeding in areas where cautery is either contraindicated or difficult.

### EXAMPLES

### Eample 1

### l Synthesis of Microgels—Hydrogel Microparticles

Materials and Methods

Microgels were synthesized from pNIPAm-AAc (p(N-isopropylacrylamide-co-acrylic-acid)) using precipitation polymerization method. The polymer chains formed microgels either with the use of 2% N,N′-methylenebisacrylamide (BIS) as a crosslinker, or without exogenous crosslinker to form ultra-low crosslinked microgels (ULC).

Sterile filtered solutions of NIPAm monomer (recrystallized from n-hexanes) and acrylic acid were mixed at 450 rpm in a reaction vessel at 70° C. before the addition of an initiator, ammonium persulfate (APS). The reaction proceeded for 6 hours before collecting the sample for purification and lyophilization. The ULC microgel composition was 95% poly(N-isopropylacrylamide) (pNIPAm)/5%AAc to provide charged groups for later encapsulation of cationic proteins. Microgel AAc groups were labeled with cadaverine conjugates of AlexaFluor dyes (AF555 or 488) via EDC/NHS chemistry for visualization with fluorescence microscopy. In order to understand whether or not particle crosslinking/structure affects fibrin morphologies, BIS crosslinked microgels were synthesized and characterized. These were formed using the same methods described previously, except N,N′-methylenebisacrylamide (BIS) at a final concentration of 2% was added to the NIPAm and AAc solution prior to the addition of APS.

Multiangle static light-scattering (3DDLS-Pro Spectrometer) was performed with a 90° setup for dynamic light scattering was additionally performed. These measurements were used for calculations of microgel volume fractions.

Results

FIGS. 2A and 2B show the findings from the static light scattering experiments, which give us insight on the ULC and BIS microgel size and structure. The hydrodynamic radius (Rₕ) is a measure of entire particle radius that can be obtained from the Stokes-Einstein equation, Rₕ=(kB T)/(6 π η D). The results demonstrate a particle diameter of approximately 1 μm for the ULC microgels. The BIS particles are only slightly smaller based on these measurements as seen in FIG. 2A. The radius of gyration (R_{g}) is the root mean squared distance between monomers within a polymer, and it gives a measure of particle internal structure. The ratio of radius of gyration to hydrodynamic radius (R_{g}/Rₕ) is a measure often used to understand a particles structure (homogenous sphere versus core-shell, etc.). Despite the low density of the ULC microgels, they approximate homogenous spheres well (dashed line in FIG. 2B) with a slight increase in core versus outer density at lower temperatures (lower R_{g}/Rₕ), and a spike near the transition temperature. This indicates that most of the particles' mass migrates to the particle periphery before homogenizing throughout the particle. BIS microgels have a slightly lower R_{g}/Rₕ value indicating a more core-shell like structure compared to the ULC microgels. Microgels were approximately 1 μm in diameter, demonstrated only slight deswelling with increasing temperature, and had structures similar to those of homogenous spheres (FIGS. 2A and 2B)

### Example 2

### Viscometry for Calculating the Volume Fraction of Microgel Suspensions

Materials and Methods

Since the particle volume fraction, Φ, is the relevant measure of concentration in colloidal suspensions, viscometry measurements to determine this quantity for both ULC and BIS crosslinked microgel suspensions were performed. An Ubbelodhe viscometer was used to measure the time it takes for microgel suspensions of various weight percentages to travel through a capillary. The time value and constant for the specific viscometer could then be used to calculate the dynamic viscosity, which could be plugged into the Einstein-Batchelor (EB) equation (η/η0=5.9Φ²+2.5Φ+1) to solve for Φ. The coefficient for the Φ² term was chosen to be 5.2 as it is known to approximate the value for soft microgel suspensions.

Viscometry was performed with the BIS crosslinked microgels to adequately control experiments with constant volume fraction. Volume fraction could also be calculated by knowing the particle size (DLS) and number density. Typically, number density measurements for particles of this size are difficult to perform due to the inability to adequately visualize them under a microscope. However, these measurements could be made using the qNano device (Izon Science Ltd), which has capabilities for quantifying number of particles/mL for colloidal particles of the size range typically below the resolution of a coulter counter or flow cytometer. Therefore, number density measurements could be made from the qNano for both the ULC and BIS microgels and compared with viscometric measurements.

Results

FIG. 3 demonstrates viscometry data from dilute suspensions of ULC microgels in 25 mM HEPES, 150 mM NaCl, and pH=7.4. Constant, k, values were obtained from fitting with the Einstein-Batchelor equation, which can be used to estimate the volume fraction of micro gels at higher concentrations as D=k*c, where c is the concentration (mg/mL) With these measurements, volume fractions could be assigned to microgel concentrations.

### Example 3

### Preparation of Fibrin Gels and Fibrin-Microgel Constructs

Materials and Methods

Human fibrinogen (CSL Behring) at 8 mg/mL (4× physiological concentrations) was used to form fibrin gels. Because fibrin polymerization and its resulting structure is dependent on multiple factors including fibrinogen, thrombin, calcium concentration, and ionic strength, the gels were formed in HEPES (N-2-hydroxyethylpiperazine-N-2-ethanesulfonic acid) CaCl₂ buffer (150 mM NaCl, 5 mM CaCl₂, 25 mM HEPES; pH 7.4) and thrombin (0.1-10 U/mL) The amount of clottable protein was determined by analyzing clot liquor protein concentration (CBQCA protein assay).

Results

Addition of microgels to fibrin gels did not significantly affect the total clottable protein incorporated into the clot (data not shown). In addition, higher fibrinogen concentrations could be used to more closely mimic concentrations represented in fibrin sealants.

### Example 4

### Structural Analysis of Fibrin-Microgel Constructs

Materials and Methods

Matrices with and without microgels were formulated as described above and polymerized for one hour between a glass slide and no. 1 coverslip. Laser scanning confocal microscopy (LSM 700, Carl Zeiss, Inc.) with a 63× oil immersion objective (NA, 1.4) was used to visualize fibrin matrices using 5% AlexaFluor 647-labeled fibrinogen and AF555 or AF488-microgels. Gels were imaged at least 25 μm above the glass interface to avoid heterogeneities in the network near contact with the glass surface.

Results

Interestingly, as opposed to homogenous distribution throughout the network in between fibrin fibers, the microgels formed pockets within these fibrin constructs. The pockets that are formed were much larger than individual microgels, and it was confirmed that the microgels sequester into these discrete pockets. The pocket sizes of the construct were calculated using custom in house generated MATLAB network quantification from the confocal images, and the results are presented in Tables 3-9.

### Example 5

### Microgel Volume Fraction within Pockets

Materials and Methods

In order to understand the physical environment the cell encounters when interacting with a microgel pocket, the volume fraction of microgels within the discrete domains was quantified. To do this, data acquired from viscometry measurements was used to determine the initial volume fraction of microgels within the polymerizing gel. Next, image analysis of confocal z-stacks was performed using in house built MATLAB and ImageJ plugins to volume available for microgels. From this, a new volume fraction for the microgel pockets was calculated based on the change in available volume after fibrin polymerization. Alternatively, the area occupied could also be acquired from confocal z-stacks of fluorescently labeled microgels within fibrin constructs; giving a direct measurement of microgel volume fraction in the pockets and will allow us to determine whether or not labeled particles result in different fibrin morphologies and hence different microgel packing inside the pockets.

Results

Values obtained from this analysis inform of the type of environment the cells are interacting with when they encounter a microgel pockets. The volume fractions and phase behavior of microgel suspensions have been extensively studied and these estimated values correspond to a range of colloidal fluids to glasses.

### Example 6

### Pocket Formation is Dependent on Polymerization Kinetics

In order to determine the effects of microgels on fibrin polymerization and understand the formation of the pocket structures, polymerization studies were performed on the confocal microscope stage to assay network formation in real time. These experiments also gave insight on understanding how matrix-microgel composites would perform if used for applications where in situ gelation is necessary. Thrombin concentration was modulated to determine the effects of polymerization kinetics on the resulting network structure.

Results

Data from experiments comparing BIS and ULC microgels suggest that BIS crosslinked microgels form less interconnected pockets. However, through modulating thrombin concentration, it was determined that the pocket formation is dependent on the kinetics of polymerization.

### Example 7

### Microgels Do Not Affect the Viscoelastic Properties of Fibrin Matrices and Form an Interconnected Network Within Fibrin

Materials and Methods

In order to study construct mechanics, a Physica MCR 501 cone and plate rheometer (Anton-Paar) was used (2.014° cone angle, and 24.960 mm tool diameter) to measure the viscoelastic properties of fibrin matrices. Both frequency (0.01Hz-10 Hz measuring 6 pts/decade, 0.5% constant strain) and strain sweeps (0.1-100% strain measuring 6 pts/decade, 1 Hz constant frequency) were performed to record the storage (G′) and loss modulus (G″) of at least 6 gels from each group.

To characterize the morphology of the pockets within matrix-microgel composites, the confocal images of the matrix-microgel composites formed with 8 mg/ml fibrin, 1 U/ml thrombin, and Φ=0.028, Φ=0.056, Φ=0.112, or Φ=0.168 microgels were analyzed. The crucial part of the analysis was associated with obtaining the skeleton of the structure, which consisted of a central line having the same topology, branching, and overall shape of the initial structure. The skeleton was calculated by solving the eikonal equation and detecting all its singularities, which form a set that correspond to the seek skeleton. The eikonal equation was solved using the fast marching method, which gave very accurate results in cartesian coordinates. From there the arc length of the skeleton was extracted. Also, the connectivity of the sample was quantified. In particular, the number and location of the branches within the skeleton, as well as the branching number of every branch point and the average distance between branches was calculated.

Results

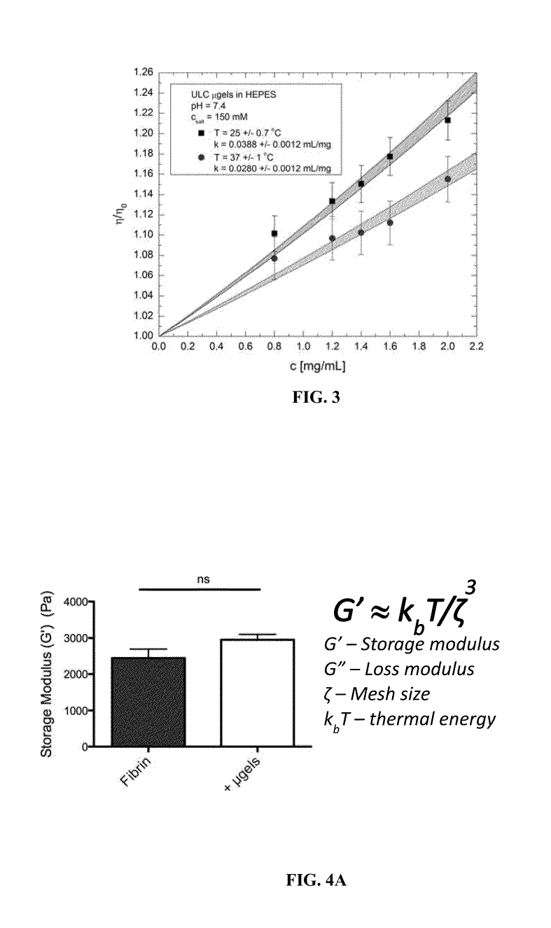

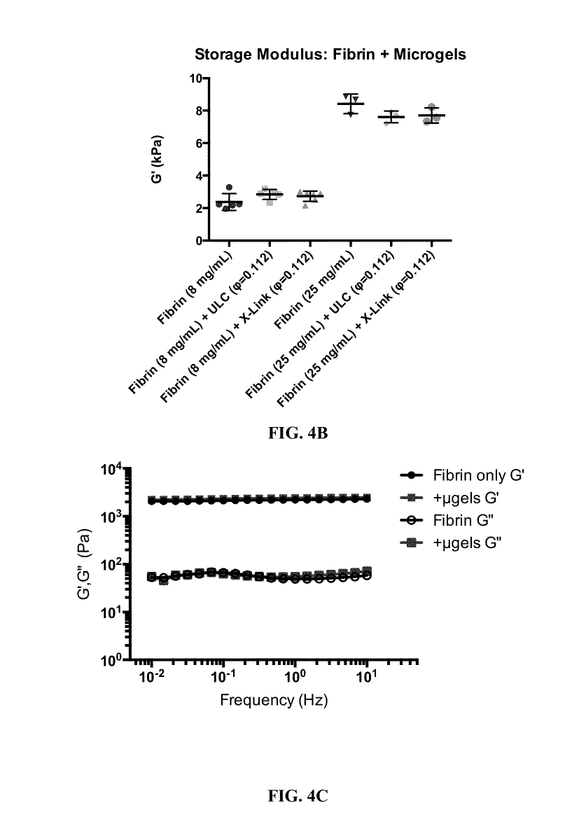

Studies on 8 mg/mL fibrin gels with and without microgels have demonstrated no statistically significant difference in the storage and loss moduli (FIGS. 4A, 4B and 4C).

Microgels formed an interconnected network within fibrin and the morphology of this network was modulated by microgel concentration or thrombin concentration. Tables 1-4 characterize the morphology of the pockets formed within matrix-microgel composites of different microgel volume fractions. Tables 5-7 characterize the morphology of the pockets formed within matrix-microgel composites formed with different thrombin concentrations.

TABLE 1
Volume percent and average radius of pockets
formed with Φ = 0.028 microgels.

| Volumes | Average Radius (μm) | Volume % |
|---|---|---|
| 1 | 4.32 | 0.090 |
| 2 | 3.83 | 0.089 |
| 3 | 3.62 | 0.071 |
| 4 | 4.43 | 0.039 |
| +280 others | | |

TABLE 2
Volume percent and average radius of pockets
formed with Φ = 0.056 microgels.

| | Average | |
|---|---|---|
| Volumes | Radius (μm) | Volume % |
| 1 | 3.31 | 0.045 |
| 2 | 3.31 | 0.023 |
| 3 | 4.48 | 0.021 |
| 4 | 4.21 | 0.019 |
| +265 others | | |

TABLE 3
Volume percent and average radius of pockets
formed with Φ = 0.112 microgels.

| | Average | Volume |
|---|---|---|
| Volumes | Radius (μm) | % |
| 1 | 4.5 | 0.916 |
| 2 | 3.41 | 0.017 |
| 3 | 5.16 | 0.013 |
| 4 | 4.1 | 0.009 |
| +8 others | | |

TABLE 4
Volume percent and average radius of pockets
formed with Φ = 0.168 microgels.

| | Average | Volume |
|---|---|---|
| Volumes | Radius (μm) | % |
| 1 | 4.36 | 0.998 |
| 2 | 4.62 | 0.0007 |
| 3 | 5.33 | 0.0007 |
| 4 | 3.95 | 0.0005 |

TABLE 5
Volume percent and average radius of pockets formed
with 0.1 U/ml of thrombin (microgels Φ = 0.112).

| | Average | |
|---|---|---|
| | Radius (μm) | Volume % |
| 1 | 5.23 | 0.976 |
| 2 | 4.36 | 0.013 |
| 3 | 3.94 | 0.003 |
| 4 | 1.23 | 0.002 |
| +6 others | | |

TABLE 6
Volume percent and average radius of pockets formed
with 1 U/ml of thrombin (microgels Φ = 0.112).

| | Average | |
|---|---|---|
| | Radius (μm) | Volume % |
| 1 | 4.5 | 0.916 |
| 2 | 3.41 | 0.017 |
| 3 | 5.16 | 0.013 |
| 4 | 4.1 | 0.009 |
| +13 others | | |

TABLE 7
Volume percent and average radius of pockets formed
with 10 U/ml of thrombin (microgels Φ = 0.112).

| | Average | |
|---|---|---|
| | Radius (μm) | Volume % |
| 1 | 3.47 | 0.9983 |
| 2 | 3.20 | 0.0008 |
| 3 | 6.23 | 0.0005 |
| 4 | 5.18 | 0.0004 |

To determine the mechanism of formation of these microgel assemblies, an imaged polymerization in real-time using fluorescently labeled fibrinogen and microgels was performed. The microgels were homogenously dispersed prior to polymerization and were driven to form packed domains with the addition of thrombin. Further, the size and architecture of these domains could be modulated through changing microgel concentration or thrombin concentration. Additionally, increasing thrombin concentration resulted in smaller diameter microgel domains Conversely, lower thrombin concentrations resulted in the formation of larger clustered microgel assemblies. This suggests that the assembly results from the polymerization dynamics; if fast, the microgels do not have time to rearrange and the resultant pockets are small, while if slow, the microgels can gradually be driven together due to fibrin polymerization.

When utilized at high volume fractions, microgels formed unique connected structures within fibrin gels (as opposed to being homogenously dispersed).

If volume fraction is controlled, similar structures were formed in fibrin irrespective of microgel crosslinking. Fibrin mechanical properties as measured with rheology are not diminished by the presence of microgels. There is a critical concentration of microgels (between microgel Φ of 0.056 and 0.112) at which a main connected volume is created within the composite networks.

### Example 8

### Alginate Polymerization with Microgels Produces Similar Results with Alternate Mechanism

Materials and Methods

Sodium alginate (or RGD modified sodium alginate) at 2%, 1%, or 0.5% in medium, was mixed together with 4 mg/ml (Φ=0.112) of ULC microgels. Following a depletion interaction, the mixture was allowed to cross-link through the addition of a divalent cation (calcium sulfate). The resulting alginate-microgel composites were studied for microgel pocket network formation as presented in Example 5.

Results

Alginate-microgel composites had similar pocket architecture as that observe for fibrin-microgel composites, indicating that microgel networks could be generated through alternative mechanisms.

A 1% solution of the anionic polysaccharide alginate was mixed with a microgel solution and imaged on a confocal microscope. Prior to gelation, effects of depletion were observed, i.e. clustering of micro gels induced from local alginate polysaccharide chains. This phenomenon was not observed in fibrinogen solutions containing microgels prior to polymerization, likely due to the difference in structure of the two precursors (fibrinogen—stiff rod, alginate—more flexible larger random coil). Alginate concentration was decreased incrementally and at concentrations below 0.5%, depletion was no longer observed. Using the Asakura-Oosawa model and calculating the theoretical osmotic pressure, volume fraction of alginate (depletants), in addition to the resulting microgel interaction potential, this hypothesis was supported. Thus, when microgels were mixed with high molecular weight alginate solutions, pre-clusters of microgels formed that then could be immobilized upon initiation ionic crosslinking of the network with the addition of Ca²⁺. Alginate concentration was the key parameter in this example, as it determined the depletion forces and drove the cluster formation and percolation.

### Example 9

### Cell Spreading in Matrix-Microgel Composites—Microgels Increase Cell Spread Area and Elliptical Factor, While Decreasing Circularity

The assembly of clustered microgel domains, which form interconnected tunnels within the composite, is key to the observed enhanced cell and tissue phenotype. These structures are achieved either dynamically, via polymerization dynamics of the bulk polymer, or via depletion interactions and subsequent gelation. In situ assembly of these unique structures represents a simple, straightforward method for generating robust tissue integration and functional vascularization within dense polymer matrices.

Materials and Methods

Fibroblasts (NIH3T3s cultured in DMEM, 10% bovine calf serum, L-glutamine, and PenStrep) were plated at 10,000 cells/gel in 100 μL fibrin gels (cells 20% v/v) within 9 mm diameter rubber gaskets in 35 mm diameter tissue culture polystyrene dishes. The constructs were allowed to polymerize for one hour at 37° C., before additional growth media was added to the surface of the gel. For spreading assays, cells were allowed to grow and spread overnight before fixing with 4% formaldehyde and staining with phalloidin (conjugated with AlexaFluor488) for visualization of the actin cytoskeleton. Z-stacks of cells within the gels were acquired on a confocal microscope and maximum intensity projections were used for spreading analyses including cell area, circularity, and elliptical factor (major axis/minor axis).

Results

Primarily rounded cells were observed in fibrin only constructs after overnight culture. This morphology is expected within these dense polymer scaffolds where cells must degrade the matrix around them in order to spread due to the small mesh size of the network. However, when microgels are incorporated into the network, robust cell spreading is observed in a microgel concentration dependent manner. As the volume fraction of ULC microgels increases, cells lose their circularity becoming more elliptical and their overall spread area increases. This trend is also observed with the BIS cross-linked microgels within volume fraction matched regimes, but to a significantly lesser extent. These findings demonstrate that the cells are able to leverage the microgel domains in order to extend protrusions and spread in three-dimensions. Additionally, this indicates a role for microgel composition, specifically deformability or intra-domain dynamics/relaxation, in dictating cellular phenotype (both motility and morphology) within these composite materials.

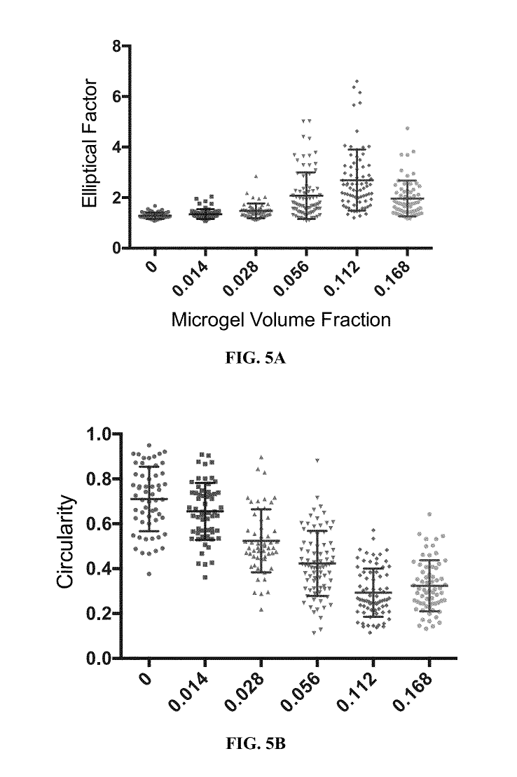

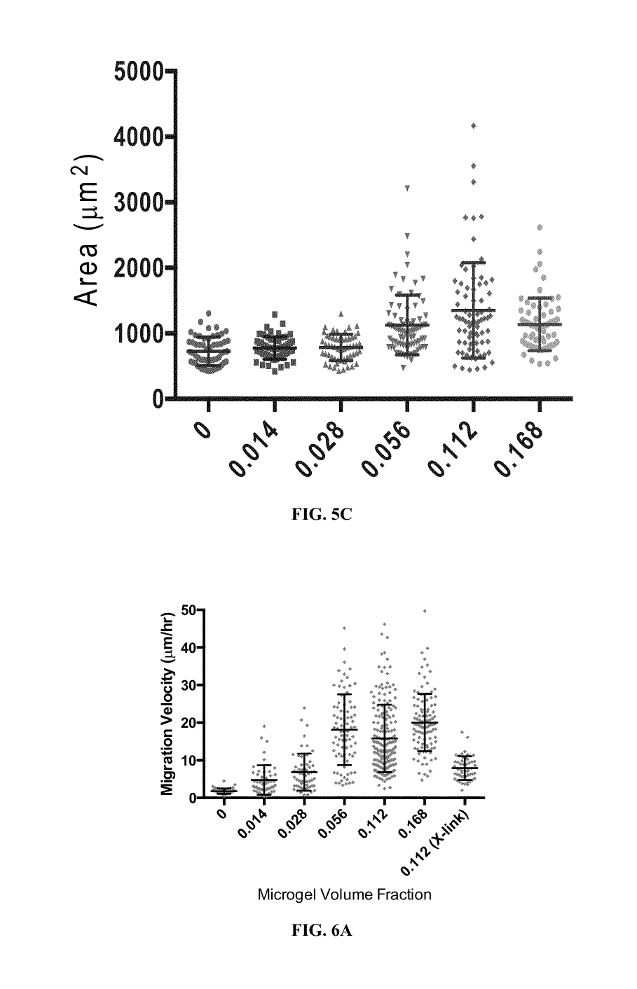

The results are presented in FIGS. 5A, 5B, and 5C. Microgels increased cell spread area and elliptical factor, while decreasing circularity. Analysis of actin-stained cells included cell area, circularity (=4*π*Area/Perimeter²), elliptical factor (major/minor axis), which were measured from at least 50 different cells in 3 different gels. (Box and whiskers plots show 95-5% confidence intervals, *** p<0.001, ANOVA).

### Example 10

### Incorporation of Microgels Into Fibrin Matrices Increases Cell Migration Velocity and Migration Distance

Materials and Methods

In order to determine if these 3D interconnected microgel tunnels could affect cellular phenotype and motility in vitro, fibroblasts were incorporated into the composite hydrogels during polymerization and were monitored over the course of 12 hours after an initial 16 hours of growth and spreading. Time-lapse microscopy of fluorescently labeled cells and manual tracking of random cell migration was performed on each of these constructs.

For motility studies, cells were stained with 10 μM Cell Tracker Dye Green CMFDA (Invitrogen) for 15 minutes in growth media according to manufacturer's instructions prior to encapsulation in gels. Cells were allowed to grow and spread overnight before beginning time-lapse experiments the next morning. Images were acquired every 10 minutes in both phase contrast and the FITC channel for 12 hours. The effect of microgels on cell migration velocity and directionality was determined. The effects of increasing microgel concentration on cell motility was analyzed from 80-100 cells per construct from 5-8 viewing fields per gel.

In order to determine the ability of NIH3T3s to invade into various gel constructs, dense cell laden gels with 10⁶ cells/mL in a 1 mg/mL fibrin gel were generated. These gels were allowed to polymerize and were then surrounded with a second gel of either fibrinogen only or matrix-microgel composites of varying relevant concentrations of microgel. After allowing the second gel to polymerize for 1 hour, cell culture media was added and the cells imaged with phase contrast microscopy at 4× magnification over the course of the experiment (7 days). ImageJ was then be utilized to measure the distance of the outgrowths into the surrounding gel from the core.

Results

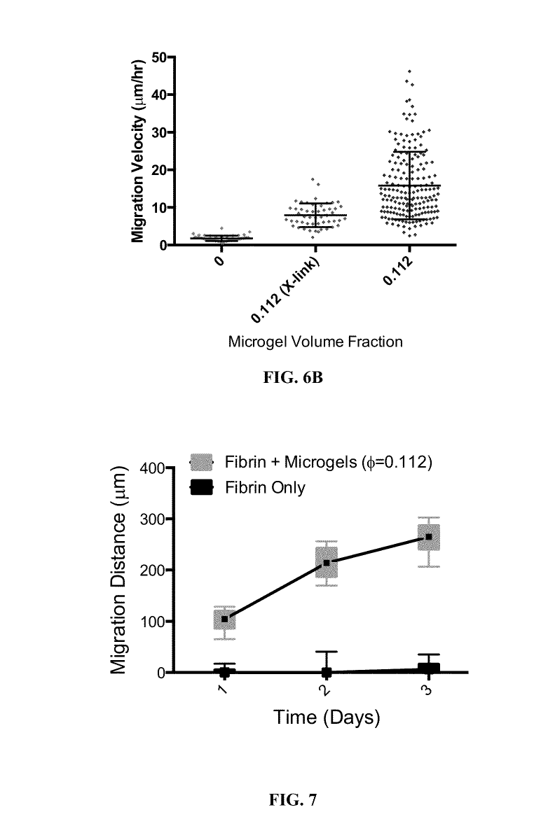

Plotting migration distance versus time, cells were seen to follow a linear trend with increasing slopes for samples containing microgels compared to fibrin only controls, indicating steady and persistent random migration through the network. An increase in average migration velocity was observed in a microgel dose-dependent manner, for example an increase was observed from an average of 6.56 μm/hr in a fibrin only control to 15.82 μm/hr with the addition of Φ=0.112 ULC microgels. Increases in average migration velocity through fibrin were also observed with the addition of equivalent volume fractions of BIS crosslinked microgels, but only reaching an average of 10.74 μm/hr, strongly suggesting that the deformability of ultra-low crosslinked microgels greatly enables cell invasion. The results for cell migration velocity are presented in FIGS. 6A and 6B. Significantly increased cell migration velocity was observed for matrix-microgel composites containing Φ of 0.056, 0.112, and 0.168 of microgel, when compared to migration velocity in constructs containing 0 of 0, 0.014, or 0.028 of microge. Velocity was calculated from individual cells encapsulated within the constructs over the course of 12 hours. Box and whiskers plots show 95-5% confidence intervals, ***p<0.05, ANOVA.

To look more specifically at infiltration, an in vitro outgrowth assay was performed to monitor migration of cells from a dense cell pellet into an outer fibrin gel with or without microgels. Over the course of three days, significant migration (hundreds of microns) into the outer gel containing ULC microgels was observed compared to the fibrin only gel, which maintained a strict unchanged border. Results of migration distance are presented in FIG. 7. A. statistically significantly outgrowth into matrix-microgel composites containing 8 mg/ml fibrin and Φ=0.112 microgels was seen at every time point compared to that into the fibrin-only control.

### Example 11

### Subcutaneous Implantation of Matrix-Microgel Composites

Materials and Methods

Constructs containing fibrin and ULC microgels (Φ=0.112) or only fibrin at 8 mg/ml, 25 mg/ml or 2.5 mg/ml, were polymerized in polycaprolactone (PCL) nanofiber meshes. Four constructs were implanted subcutaneously per rat, into a total of 8-10 rats, rotating the anatomical position in order to minimize artifacts of location specific differences in vascularization At the 4-week time point, the vasculature of the rats was perfused with micro-FiI contrast agent (n=8-10) and AlexaFluor488DyLight Lectin (n=3). The implants were then harvested for micro-CT and histological analyses.

Results

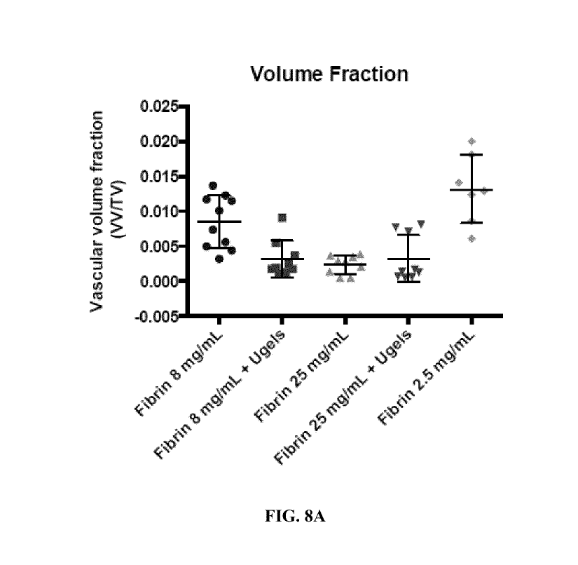

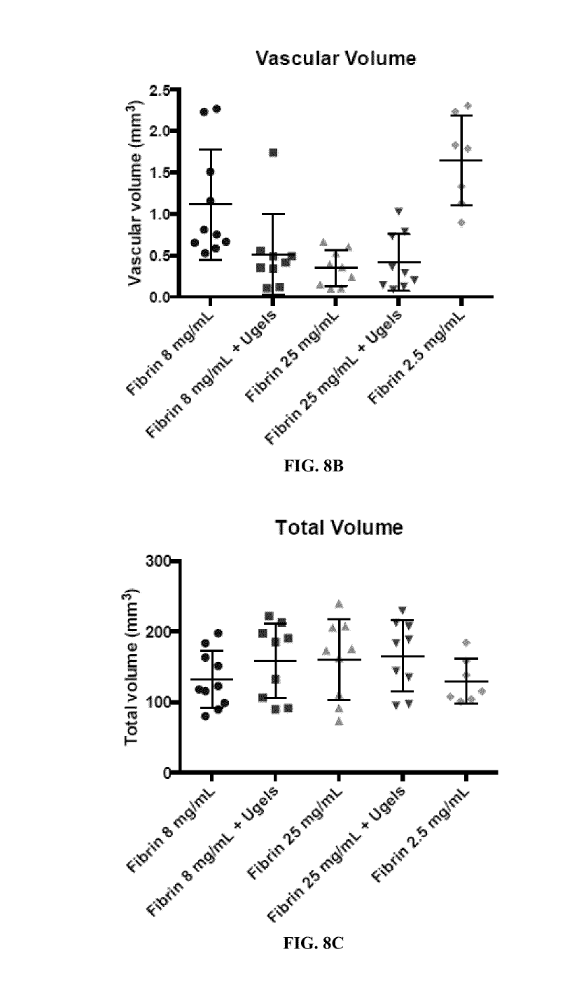

Microgels were still present within the constructs after 4 weeks. The vascular volume fraction derived from the vascular volume (VV) normalized to the total volume (TV) is presented in FIG. 8A. The vascular volume (mm³) is presented in FIG. 8B, and the total volume (mm³) is presented in FIG. 8C. Micro-CT angiography data and histological analysis of cell infiltration via H&E staining showed similar results and indicated that at high fibrin concentrations, the presence of microgels had no dramatic effect on vascularization (n=9) or ingrowth (n=7). This result is likely due to incorrect model selection as the implantation of the constructs was done at sites that have known low vascularization/angiogenic responses.

### Example 12

### Microvessel Angiogenesis in Matrix-Microgel Composites

Materials and Methods

Adipose tissue was harvested from the epididymal tissue of male sprague-dawley rats, digested into multicellular microvessel fragments (MVF), and embedded in composite gels containing either fibrin alone, or fibrin and ULC microgels at Φ=0.112. Gels with primary isolated microvessels were cultured in vitro for 10 days to allow for sprouting and neovessel formation. The samples were then fixed and stained with GSL-1 lectin to stain endothelial cells, and Hoescht for nuclei. The stained constructs were imaged on a confocal microscope to obtain 3D reconstructions of the microvessel network. The images were then analyzed and skeletonized using in house MATLAB quantification. Vessel arc length (mm), total vessel volume (mm³), and total number of branches were calculated from skeletonized images.

Results

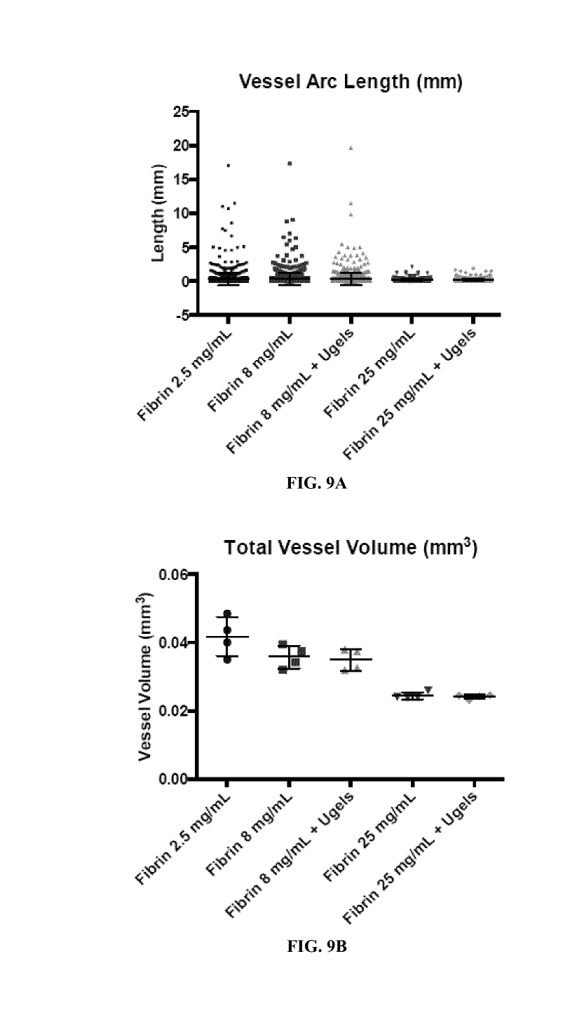

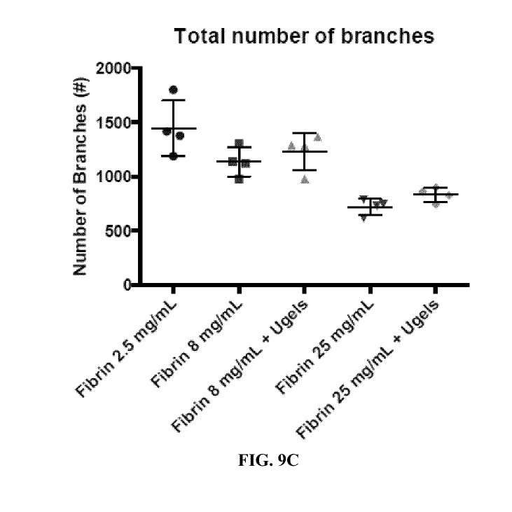

The addition of microgels did not affect cell infiltration or growth, as the vessel arc length, the total vessel volume, or the total number of branches were comparable to the constructs without microgels (FIGS. 9A, 9B and 9C). This result is also likely due to incorrect model selection.

However, these data indicate that the microgels could be used as a delivery system to incorporate agents into microgels and deliver these to the sites of interest with fibrin-microgel composites.

Although constructs used here were pre-cast, this technology could easily translate into an in situ delivery system with polymerization and self-assembly of these hybrid materials at the site of injury. This feature would be beneficial compared to other biomaterials with pre-fabricated vascular networks or those containing porogens where extensive processing steps are required prior to use of the product. Therefore, the addition of colloidal assemblies of microgels could serve as a simple addition to densely crosslinked biomaterial hydrogels for improvement in tissue integration with the host.

Unless defined otherwise, all technical and scientific terms used herein have the same meanings as commonly understood by one of skilled in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A composition comprising a polymeric matrix comprising an microgel.

2. The composition of claim 1, wherein composition has a microgel volume fraction of 0.06 to 0.50.

3. The composition of claim 1, wherein the microgel is suspended in the polymeric matrix.

4. The composition of claim 1, wherein the microgel is homogeneously dispersed within the polymer matrix.

5. The composition of claim 1, wherein the microgel(s) segregate into pockets within the polymer matrix.

6. The composition of claim 1, wherein the polymeric matrix comprises fibrin.

7. The composition of claim 1, wherein the compositions comprise cells.

8. The composition of claim 7, wherein the cells are human cells.

9. The composition of claim 1, wherein the polymer network is formed of proteins, polysaccharides, biocompatible polymers, or combinations thereof.

10. The composition of claim 1, wherein the microgel comprises poly(N-isopropylacrylamide) (pNIPAm), polyketal, poly(ethylene glycol), or poly[(oligo ethylene glycol)methacrylate] polymer, or polysaccharides, or polymerized proteins,

11. The matrix-microgel composite of claim 1, further comprising tissue repair and regeneration factors.

12. The matrix-microgel composite of claim 11 wherein the tissue repair and regeneration factors include therapeutic or prophylactic agents.

13. A method of making a polymeric matrix, the method comprising
combining a microgel or microgels with a monomer solution,
polymerizing or crosslinking the monomers to form a polymer matrix comprising the microgel, wherein the polymer matrix has a microgel volume fraction above 0.06.

14. The polymer matrix formed by the method of claim 13.

15. A method of repairing or regenerating a tissue, comprising administering to a subject in need thereof an effective amount of the composition of claim 1, wherein the composition comprises a therapeutic agent.

16. The method of claim 15, wherein the composition is seeded with human cells.

17. The method of claim 16 wherein the human cells are multipotent.

18. A method for delivering a therapeutic substance to a subject in need thereof, comprising
loading the composition of claim 1 with the therapeutic substance and administering the loaded composition to the subject, wherein the loaded composition releases an effective amount of the therapeutic substance into or onto the subject to treat the subject.

19. The method of claim 1, wherein the therapeutic substance promotes or enhances wound repair or tissue regeneration.
